(19) United States
(12) Patent Application Publication
(10) Pub. No.: US 2026/0185075 A1
(43) Pub. Date: Jul. 2, 2026

(54) SYSTEMS AND METHODS FOR PRECISION IRRADIATION OF CELLULAR CULTURES AND ANIMAL PHENOTYPE SUBJECTS

(71) Applicant: Empyrean Medical Systems, Inc., Boca Raton, FL (US)

(72) Inventors: Dirk Bartkoski, Knoxville, TN (US); Limor Erez, Netanya (IL)

(21) Appl. No.: 19/326,166

(22) Filed: Sep. 11, 2025

Related U.S. Application Data
(60) Provisional application No. 63/694,598, filed on Sep. 13, 2024.
(60) Provisional application No. 63/753,109, filed on Feb. 3, 2025.
(60) Provisional application No. 63/799,035, filed on May 2, 2025.

(51) Int. Cl. C12N 13/00; A61D 7/04; A61D 99/00; C12M 1/36; C12M 1/42

(52) U.S. Cl. CPC C12N 13/00; A61D 7/04; A61D 99/00; C12M 35/02; C12M 41/48

(57) ABSTRACT

Systems and methods for operating a cabinet irradiator. The methods comprise: receiving a sample holder in a receptacle of an XYZ stage platform of the cabinet irradiator (sample(s) is(are) disposed on the sample holder); controlling the XYZ stage platform to move the sample holder under a camera such that an optical axis is centered over a sample area of the sample holder; displaying a top-down image of the at least one sample in a first graphical user interface along with at least one virtual box having an adjustable size and being movable relative to the top-down image; receiving a user input specifying at least one area of the at least one sample that is to be irradiated via movement and or resizing of the at least one virtual box; receiving a user input selecting or defining an irradiation treatment plan; and irradiating the sample(s) in accordance with irradiation treatment plan.

### CROSS-REFERENCE TO RELATED APPLICATIONS

The present application claims priority to and the benefit of U.S. Provisional Patent Application No. 63/799,035 filed on May 2, 2025 and U.S. Provisional Patent Application No. 63/694,598 filed on Sep. 13, 2024, and U.S. Provisional Patent Application No. 63/753,109 filed on Feb. 3, 2025. The contents of the listed Provisional Patent Applications are incorporated herein by reference in their entireties.

### BACKGROUND

Cell irradiation is used for radiobiology research. Cell irradiation generally involves exposing cell cultures to radiation. The radiation can include x-rays. The effects of the radiation on the cellular cultures may then be analyzed and studied. For example, DNA damage, cell death and/or cell cycle alterations may be detected and analyzed. The radiation dose and/or exposure time may be varied during the study to, for example, develop radiotherapy protocols for improving treatment outcomes.

### SUMMARY

The present disclosure concerns implementing systems and methods for operating a cabinet irradiator. The methods comprise: receiving a sample holder in a receptacle of an XYZ stage platform of the cabinet irradiator (wherein at least one sample is disposed on the sample holder); controlling, by a processor, the XYZ stage platform to move the sample holder under a camera such that an optical axis is centered over a sample area of the sample holder; displaying, by the cabinet irradiator, a top-down image of the at least one sample in a first graphical user interface along with at least one virtual box having an adjustable size and being movable relative to the top-down image; receiving, by the processor, a user input specifying at least one area of the at least one sample that is to be irradiated via movement and/or resizing of the at least one virtual box; receiving, by the processor, a user input selecting or defining an irradiation treatment plan; and irradiating, by the cabinet irradiator, the at least one sample in accordance with the irradiation treatment plan.

The present document also concerns a system comprising a processor and a non-transitory computer readable medium. The computer readable medium comprises programming instruction(s) that when executed by the processor, cause the processor to: detect when a sample holder has been received in a receptacle of an XYZ stage platform of a cabinet irradiator (wherein at least one sample is disposed on the sample holder); control the XYZ stage platform to move the sample holder under a camera such that an optical axis is centered over a sample area of the sample holder; display a top-down image of the at least one sample in a first graphical user interface along with at least one virtual box having an adjustable size and being movable relative to the top-down image; receive a user input specifying at least one area of the at least one sample that is to be irradiated via movement and or resizing of the at least one virtual box; receive a user input selecting or defining an irradiation treatment plan; and cause the cabinet irradiator to irradiate the at least one sample in accordance with the irradiation treatment plan.

The present document also concerns a sample holder. The sample holder comprises: a planar irradiation bed comprising a plurality of apertures formed in a grid pattern; at least one accessory comprising at least one end configured to be inserted into and removed from each of the plurality of apertures; a first connector coupled to the planar irradiation bed and configured to mate with a second connector of a receptacle of an irradiation cabinet to establish an electrical connection between a circuit of the sample holder and a circuit of the irradiation cabinet; and a gas delivery tube configured to receive a gas from the irradiation cabinet and deliver the gas to a sample area of the planar irradiation bed.

The present document also concerns implementing systems and methods for operating an irradiation system. The methods comprise: receiving, by in an irradiation chamber of an irradiator, a wheel structurally supported by a stand; suspending a plurality of sample holders on an outer edge of the wheel in a manner that maintains a horizontal alignment of each of the sample holders with a surface of the irradiation chamber on which the stand sits; exposing a first sample disposed on a first sample holder of the plurality of sample holders to radiation with a uniform distribution and at a specific dose rate, the first sample holder being closest to an x-ray tube of the irradiator than other ones of the plurality of sample holders; discontinuing radiation exposure to the first sample by rotating the wheel within the irradiation chamber so as to move the first sample holder away from the x-ray tube and out of alignment with an x-ray axis; continue rotating the wheel within the irradiation chamber so as to move another second sample holder closer to the x-ray tube and into alignment with the x-ray axis; and/or exposing a second sample disposed on the second sample holder of the plurality of sample holders to the radiation with the uniform distribution and at the specific dose rate.

The present document also concerns an irradiation system. The irradiation system comprises: a vertical wheel structurally supported by a stand such that the wheel can rotate; and a plurality of sample holders suspended on an outer edge of the vertical wheel in a manner that maintains a horizontal alignment of each of the sample holders with a surface on which the stand sits. A surface-to-skin distance parameter for an irradiation treatment plan is defined by a distance between an x-ray tube and a sample disposed in a top most one of the sample holders.

### BRIEF DESCRIPTION OF THE DRAWINGS

The present solution will be described with reference to the following drawing figures, in which like numerals represent like items throughout the figures.

FIGS. 1-2 provide illustrations of a cabinet irradiator.

FIG. 3 provides an illustration showing a block diagram of the cabinet irradiator shown in FIGS. 1-2.

FIG. 4 provides an illustration of another architecture for a cabinet irradiator.

FIGS. 5-7 provide graphs of a radiation profile for an x-ray tube. With regard to FIG. 7, profiles are measured using the ionization chamber in water (line) and calculated using Monte Carlo (symbols) for 4×4 (a), 10×10 (b), and 20×20 cm² (c) and depths of 1 cm and 5 cm (circle and diamond respectively) for the 2250-kVp, 17-mA, 0.35-mm Cu beam. The data is normalized to the central axis value at 1-cm depth.

FIGS. 8-9 each provides an illustration that is useful for understanding how a source-to-skin distance (SSD) is determined.

FIG. 10 provides an illustration showing a partial front view of components disposed or otherwise positioned in an irradiation chamber.

FIG. 11A provides a perspective view of a sample holder with a sample disposed thereon.

FIGS. 11B-11C each provides a side view of a sample holder.

FIGS. 12A-12E (collectively referred to as “FIG. 12”) provide a flow diagram of an illustrative method for operating a cabinet irradiator and/or performing an irradiation process.

FIGS. 13A-13J (collectively referred to as “FIG. 13”) provide illustrative graphical user interfaces for a cabinet irradiator.

FIG. 14 provides a perspective view of a carousel for samples.

FIG. 15 provides a flow diagram of an illustrative method for operating an irradiation system.

FIG. 16 provides a block diagram of an illustrative architecture for a computing device.

### DETAILED DESCRIPTION

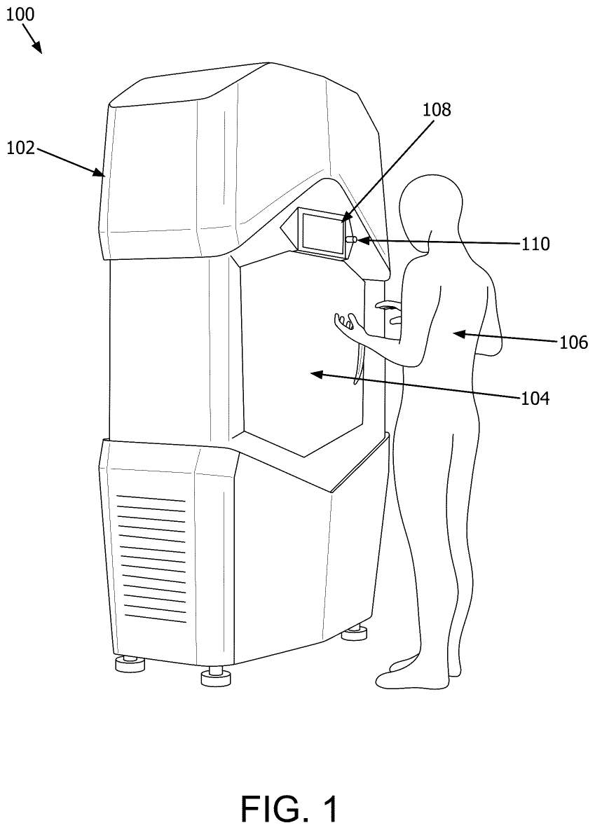

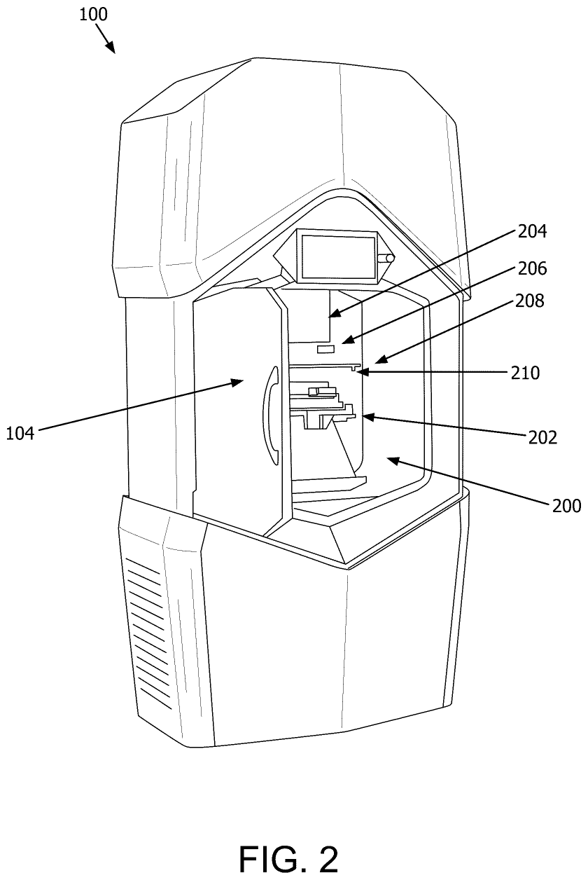

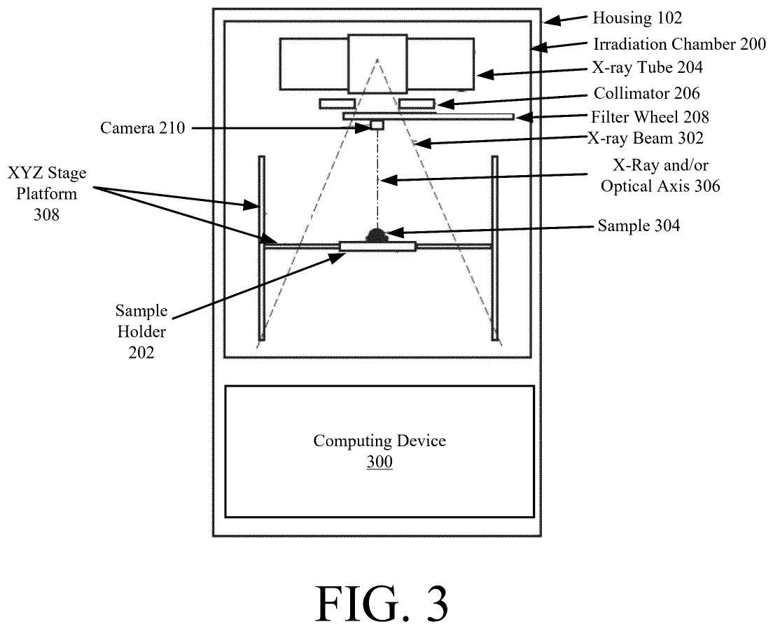

FIGS. 1-2 provide illustrations of a cabinet irradiator 100 in accordance with the present solution. FIG. 3 provides an illustration showing a block diagram of the cabinet irradiator 100. Cabinet irradiator 100 is generally configured to expose sample(s) 304 to x-rays for various purposes such as sterilization, decontamination and/or material modification. Sample(s) 304 can include, but are not limited to, cellular cultures, animal phenotype subjects, and/or objects (e.g., medical devices, tools, and/or other products). The objects can be formed of any suitable material such as metal(s) and/or plastic(s). Cabinet irradiator 100 is configured to produce x-rays using a high-voltage electron beam that collides with a target material. The target material can include, but is not limited to, copper or tungsten.

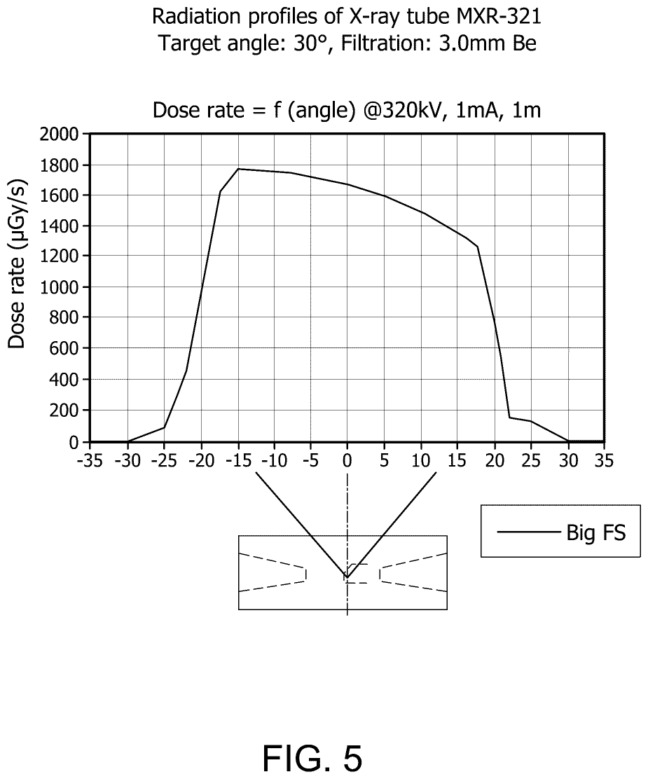

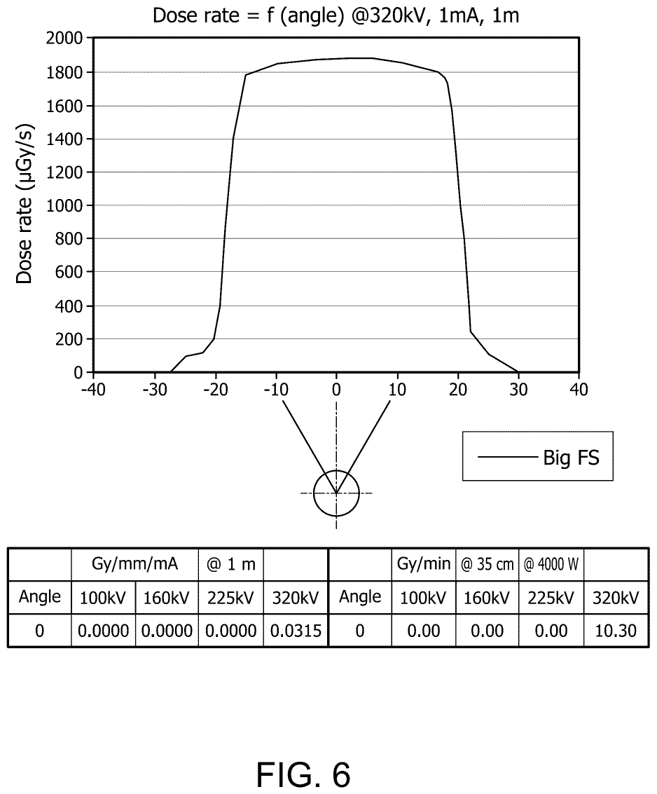

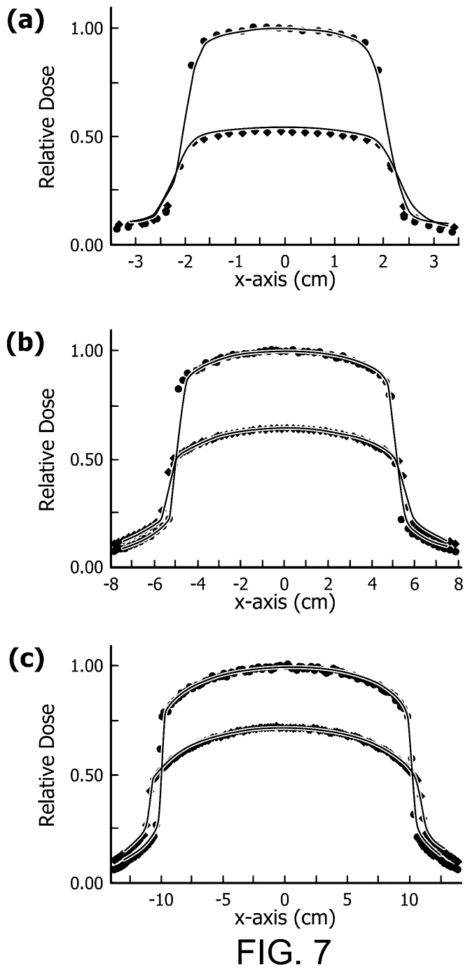

Cabinet irradiator 100 comprises a housing 102 with a door 104 to allow an individual 106 access to an internal irradiation chamber 200. Irradiation chamber 200 is sized and shaped to receive and structurally support sample holder(s) 202. An x-ray tube 204 and a collimator 206 are disposed inside the irradiation chamber 200. Any known or to be known x-ray tube and/or collimator can be used here. For example, x-ray tube 204 can include, but is not limited to, a commercially available x-ray tube with product number MXR-321. Graphs are provided in FIGS. 5-7 of a radiation profile for the MXR-321 x-ray tube. X-ray tube 204 comprises a vacuum tube that produces x-rays by accelerating electrons and causing them to impact a target material. Collimator 206 comprises a device that shapes or restricts an x-ray beam 302 into parallel or less divergent rays. Collimator 206 may be used to create an x-ray beam 302 with a specific shape and/or size. X-ray beam 302 has an x-ray axis 306, i.e., a reference line that describes a central path of the x-ray beam 302 as it passes through the irradiation chamber 200.

A filter wheel 208 is also disposed inside the irradiation chamber 200. The filter wheel 208 comprises a plurality of different filters that are spaced apart relative to each other. The filters can include, but are not limited to, a 0.5-mm copper filter, a 0.75-mm tin, 0.25-mm copper, 1.5-mm aluminum composite filter, and/or a 2.25-mm copper filter. The filter wheel 208 is rotatable or otherwise movable such that the filters can be selectively rotated or otherwise moved within the cabinet irradiator 100. In this regard, motors, gears and other mechanical and/or electro-mechanical mechanism(s) may be provided with the irradiation chamber 200 to facilitate rotation and/or other movement of the filter wheel 208. As such, the filters have variable positions that are configured to generate defined x-ray qualities for calibration of detectors or dosimeters in radiological qualities. Positions of the filters may be controlled by computing device 300.

Camera(s) 210 may be provided inside irradiation chamber 200 to capture images and/or video of sample(s) 304 while in the irradiation chamber 200 prior to, during, and subsequent to an irradiation process. A camera 210 may be mounted on the rotating filter wheel 208 below the collimator 206. As such, the camera 210 may be transitioned between a plurality of different positions relative to other components inside the irradiation chamber 200. Other camera(s) may be provided inside the irradiation chamber 200 so as to have stationary positions and/or variable positions relative to other components of the irradiation chamber 200. Any known or to be known camara(s) can be used here. XYZ stage platform 308 provide precise positioning systems that allow for accurate movement of the sample 304 in three dimensions-X (horizontal), Y (horizontal), and Z (vertical).

Operation of the cabinet irradiator 100 may be controlled by a computing device 300. Computing device 300 can include a display screen 108 or other user-interface device(s) 110 to allow the individual 106 to configure, start, monitor and/or stop the irradiation process. The display screen 108 may be a touch screen. The other user-interface device(s) 110 can include, but is not limited to, push button(s), rotary knob(s), and/or switch(es). Particulars of the irradiation process and/or control thereof will become evident as the discussion progresses.

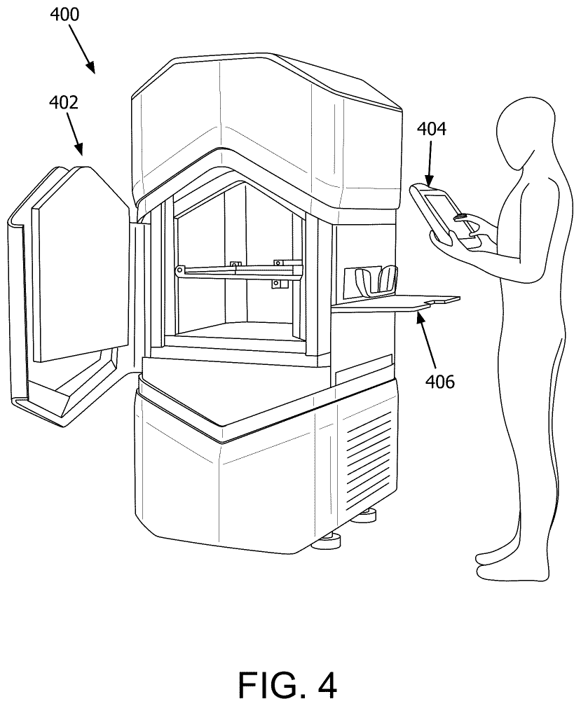

The present solution is not limited to the architecture of cabinet irradiator 100 shown in FIGS. 1-3. Another architecture 400 for the cabinet irradiator is shown in FIG. 4. This architecture 400 has a door 402, a repositionable touch screen 404, and a drop-down table 306. The repositionable touch screen 404 can include, but is not limited to, a tablet, an iPad, a smart phone, or other portable computing device. Accessory storage may also be provided with architecture 400.

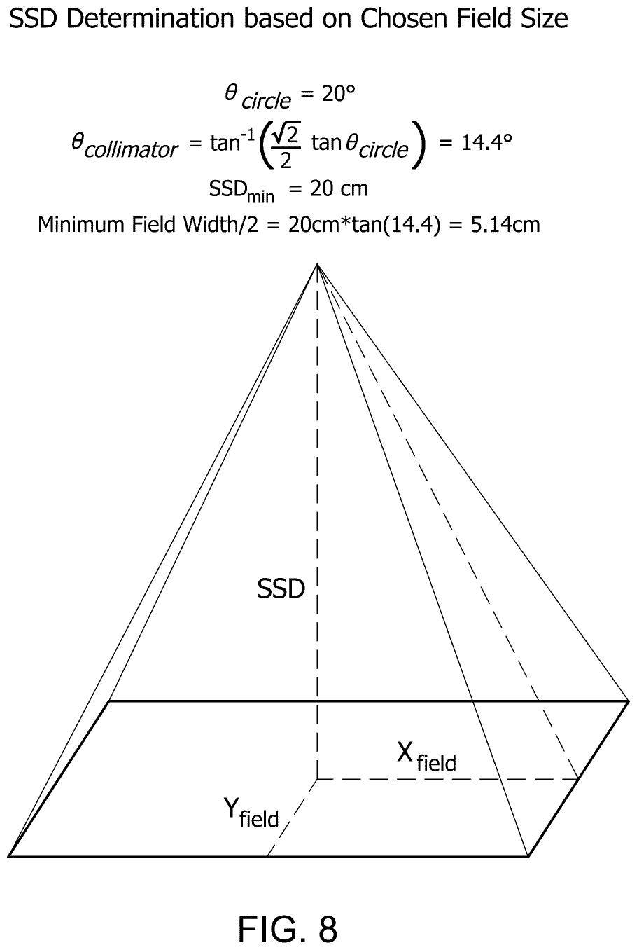

FIG. 8 provides an illustration showing how an SSD is determined by individual 106 and/or computing device 300 based on a chosen field size. SSD is the distance between x-ray tube 204 and the surface of the sample 304 where the x-ray beam 302 enters. SSD is an important factor for dose calculation. In some scenarios, a minimum field width for the x-ray beam 302 may be computed in accordance with the following mathematical equations (1)-(3). The minimum field width may refer to the smallest size of the x-ray beam 302 that can be used during an irradiation process without the use of collimator 206.

$${θ}_{circle} = 20 ° \qquad ( 1 )$$

$${θ}_{collimator} = {tan}^{- 1} ( \frac{\sqrt{2}}{2} tan {θ}_{circle} ) = 14.4 ° \qquad ( 2 )$$

$$\begin{matrix}SSDmin = 20 cm \\ Minimum Field Width / 2 = 20 cm * tan ( 14.4 ) = 5.14 cm\end{matrix} \qquad ( 3 )$$

The present solution is not limited to the field width defined by mathematical equations (1)-(3).

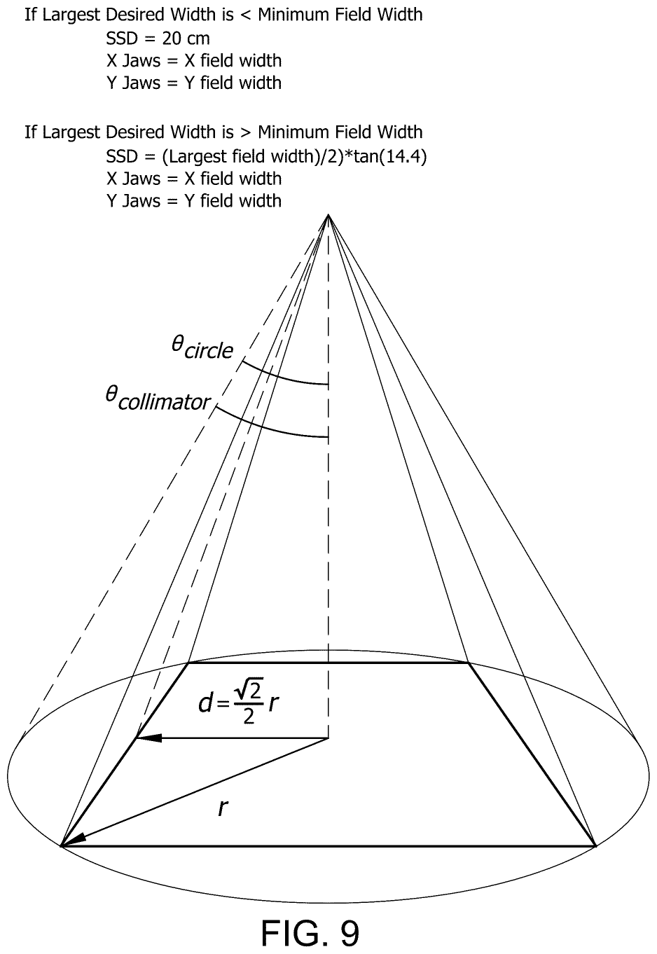

FIG. 9 provides an illustration showing how to compute the SSD when the desired field width is greater than the minimum field width. This computation may be defined by the following mathematical equation (4).

$$SSD = ( ( field width ) / 2 ) / * tan ( 14.4 ) \qquad ( 4 )$$

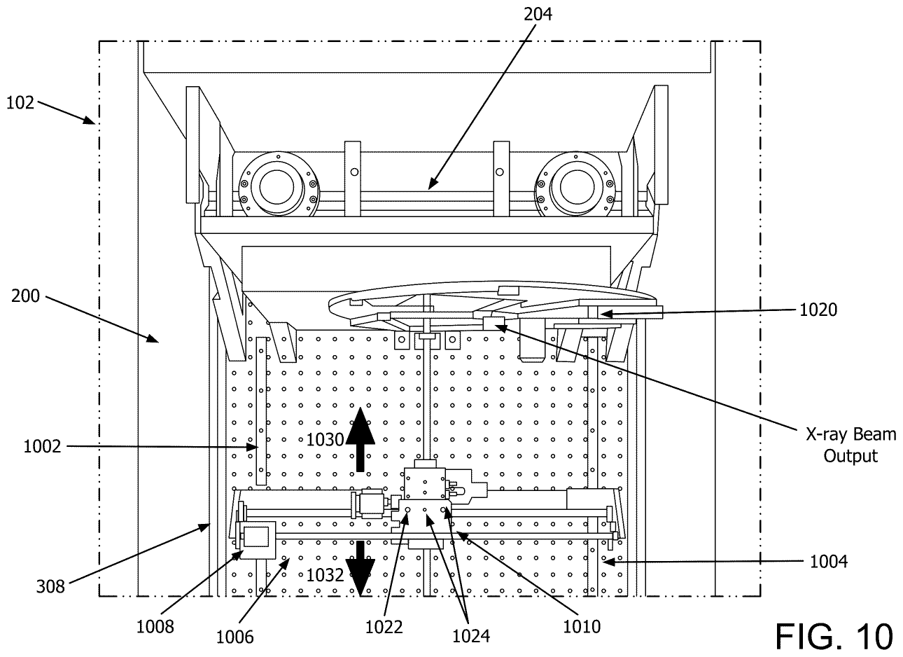

FIG. 10 provides an illustration showing a portion of the irradiation chamber 200. The XYZ stage platform 308 comprises a horizontal structure 1006 coupled to vertical tracks 1002, 1004. Motors and/or gears 1008 are provided with the horizontal structure 1006 to facilitate the vertical travel of the horizontal structure 1006 in directions 1030, 1032 along tracks 1002, 1004. Any known or to be known mechanical and/or electro-mechanical technique for causing movement of an object can be used here. Component(s) 1008 is(are) controlled by computing device 300.

A connector 1010 is provided with horizontal structure 1006. Connector 1010 comprises electrical ports 1024 to facilitate an electrical connection between the XYZ stage platform 308 and a sample holder 202. The sample holder 202 includes electronic components that may be powered by a power supply of the cabinet irradiator 100. The electronic components can include, but are not limited to, sensor(s), processor(s), memory(ies), and/or motor(s). Data and/or other information (e.g., instructions) may be communicated between the electronic components of the sample holder and the computing device 300 via the electronic connection. Connector 1010 also comprises a gas port 1022 to facilitate a supply of anesthesia or other gas or fluid from the cabinet irradiator 100 to the sample holder 202.

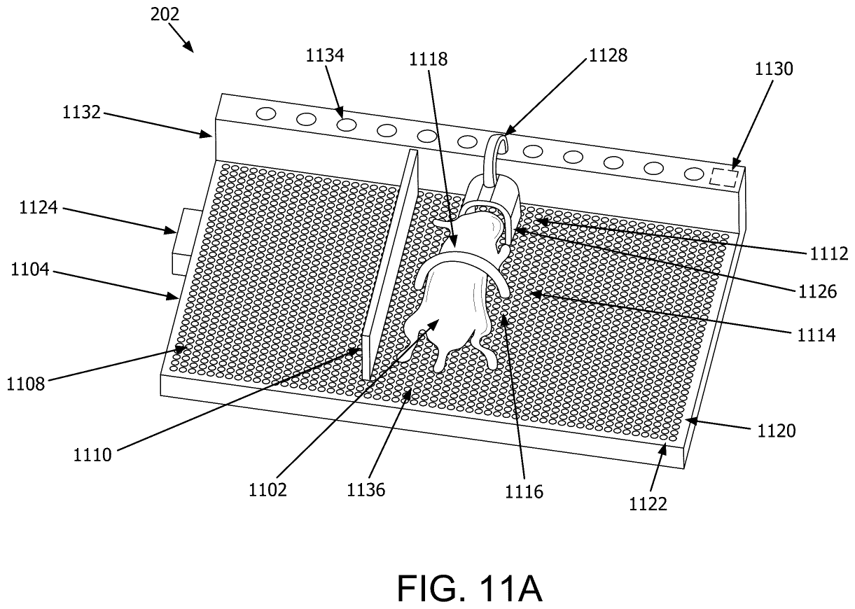

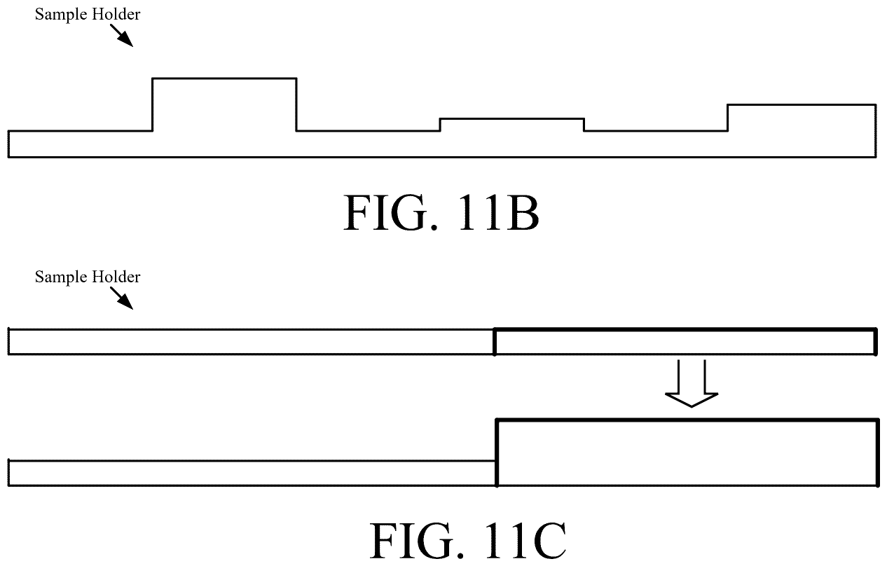

FIG. 11 provides an illustration showing an architecture for the sample holder 202. Sample holder 202 is configured to structurally support and retain samples in certain positions for irradiation. Sample holder 202 is preconfigured with the system such that each of the sample holder's dimensions and characteristics are known by the system. The dimensions can include, but are not limited to, length, width, and/or thickness. The characteristics can include, but are not limited to, material, electrical properties (e.g., resistance), thermal properties (e.g., thermal conductivity), and/or radiation characteristics (e.g., spectral radiative properties—reflectivity, absorptivity and transmissivity). Sample holder 202 may comprise, for example, glass and/or plastic.

The sample holder 202 may have a barcode or other information printed or otherwise disposed thereon that can be scanned or otherwise acquired. Camera(s) 210 may be used to scan or acquire information from sample holder. Additionally or alternatively, some or all of the information may be input by individual 106 via user interface device(s) 110. The scanned and/or other information may be used to access a look-up table (LUT) or datastore to retrieve the sample holder's dimensions and/or characteristics. The scanned and/or other information can include, but is not limited to, a sample holder identifier and/or a sample holder type identifier. The scanned information may be used as an index for retrieving data stored from the LUT and/or datastore.

Multiple sample holders may be provided for use with the cabinet irradiator 100. The sample holders can have the same or different dimensions and/or characteristics. As such, sample holder 202 may be interchangeable with other sample holders.

Sample holder 202 is designed to allow the individual 106 to position, couple and/or restrain one or more samples 100. Sample 1102 is shown as comprising a mouse. The present solution is not limited in this regard. Sample 1102 can include any other type of sample selected in accordance with a given application. The sample may be a living or non-living sample.

The coupling and/or restraining of the sample can be achieved using restraining member 1114. Restraining member 1114 may have a generally curved shape and/or may be formed of any suitable rigid, semi-rigid or flexible material (e.g., plastic and/or rubber). The curved shape can include, but is not limited to, a C-shape, a U-shape, and/or a cup shape. Restraining member 1114 may be transparent so that it does not obstruct an onlooker's view of the sample and/or hide a portion of the sample in captured video(s) and/or image(s). The two free ends 1116 of restraining member 1114 may be configured to snap, frictionally engage, or otherwise releasably connect into apertures 1108 formed in a planar irradiation bed 1104 of the sample holder 202. Any known or to be known snap-fit, friction-fit and/or other releasable-connector configurations can be used here. For example, each free end 1116 may comprise a protruding post with a diameter smaller than the diameter of a center portion 1118 of the restraining member 1114. The post may or may not comprise a flange, lip, detent and/or dimple. The present solution is not limited to the particulars of this example.

Apertures 1108 may be equally spaced apart and arranged in a grid pattern comprising a plurality of rows 1120 and a plurality of columns 1122. This grid arrangement of the apertures allows for the selective coupling of one or more samples to the sample holder and/or allows for each sample to be placed and retained in a variety of positions relative to the irradiation bed 1104. The grid arrangement of the apertures may facilitate the translation of sample position coordinates to corresponding position(s) and/or shift(s) of the XYZ stage platform that place the center of a sample area to be irradiated in line or alignment with the x-ray axis 306.

Other accessories may also be provided with the sample holder 202. Each accessory may comprise legs that may be inserted into and removed from the apertures 1108 such that it may be securely and removably coupled to the irradiation bed 1104. The grid of apertures and accessories are designed such that accessory pieces may be easily repositioned and reconfigured over the surface of the sample holder 202 allowing multiple configurations of the sample irradiation surface. The accessories can include, but are not limited to, restraining member(s) 1114 for holding small animal anatomy or limbs in various positions, shielding plate(s) 1110 to reduce or shield secondary scatter radiation from traveling into adjacent areas, gas mask(s) 1112, and/or other environmental control component(s). The environment control component(s) can include, but are not limited to, devices (e.g., heater(s), cooler(s), etc.) and/or humidity absorber(s). One or more of the environment control component(s) may be standalone component(s) that are separate and apart from the shielding plate(s) 1110, gas mask(s) 1112 and/or restraining member(s) 1114. Additionally or alternatively, one or more of the environment control component(s) may be integrated in the shielding plate(s) 1110, gas mask(s) 1112 and/or restraining member(s) 1114.

The gas mask 1112 comprises a mask portion 1126 and a gas delivery tube 1128 for delivering anesthesia and/or other gas to a sample. The other gas can include, but is not limited to, oxygen. The mask portion 1126 is sized and/or shaped to ensure that the anesthesia and/or other gas remain(s) in proximity to at least a portion of the sample (e.g., a living sample's face for inhalation). For example, the mask portion 1126 has a generally curved shape. The curved shape can include, but is not limited to, a U-shape. The gas delivery tube 1128 has a proximal end that is connected to the mask portion 1126 and extends out from the mask portion 1126. A free distal end of the gas delivery tube 1128 is configured to connect to a sidewall 1132 of the sample holder 202 via tube connection port(s) or terminal(s) 1134. Tube connection port(s) or terminal(s) 1134 may comprise valve(s) or other means for controlling the passage of fluid to the gas mask 1112. The valve(s) or other means may be enabled or activated when a gas delivery tube 1128 is inserted in, coupled to, and/or connected to the respective tube connection port(s) or terminal(s) 1134. A gas delivery tube (not visible in FIG. 11) is also disposed inside the sidewall 1132 and connected to connector 1124 so that anesthesia and/or gas may be supplied to the sample from the cabinet irradiator 100 prior to, during, and/or subsequent to an irradiation process. Gas mask 1112 may be formed of any suitable material such as a plastic or rubber. Gas mask 1112 may be transparent so that it does not obstruct an onlooker's view of the sample and/or hide a portion of the sample in captured video(s) and/or image(s).

Sample holder 202 is also configured to interface with the cabinet irradiator 100. In this regard, sample holder 202 comprises a connector 1124 configured to mate with the connector 1010 of the XYZ stage platform 308. Connector 1124 comprises electrical terminals (not visible in FIG. 10) sized and shaped to be received in electrical ports 1024 of connector 1010. Any known or to be known mating connectors can be used here. Information may be communicated from a circuit 1130 of the sample holder 202 to the computing device 300 of the cabinet irradiator 100. This information can include, but is not limited to, a unique identifier for the sample holder 202, geometric dimensions of the sample holder 202, characteristics of the sample holder 202, and/or sensor data. The sensor data can be generated by sensors of the circuit 1130. The sensors can include, but are not limited to, temperature sensor(s), humidity sensor(s), oxygen sensor(s), anesthesia detection sensor(s), and/or other environmental sensor(s). Circuit 1130 may also comprise processor(s) and/or datastore(s). Circuit 1130 may comprise a local power source (e.g., a rechargeable battery, an energy harvesting circuit) and/or may be powered by a power source of the cabinet irradiator.

In some scenarios, the sample holder 202 may be designed to compensate for inherent x-ray beam non-uniformity without the reduction of dose rate arising from the use of flattening filters. For example, sample holder 202 may be designed such that its height varies thereacross (as shown in FIG. 11B) and/or such that its height is adjustable at various locations there along (as shown in FIG. 1C). In the latter case, the height of a portion of the sample holder 202 is adjusted so that the sample placed thereon has a modified SSD as compared to other samples placed on the sample holder 202. This may allow dose rates for samples across the sample holder's surface to remain constant or approximately constant.

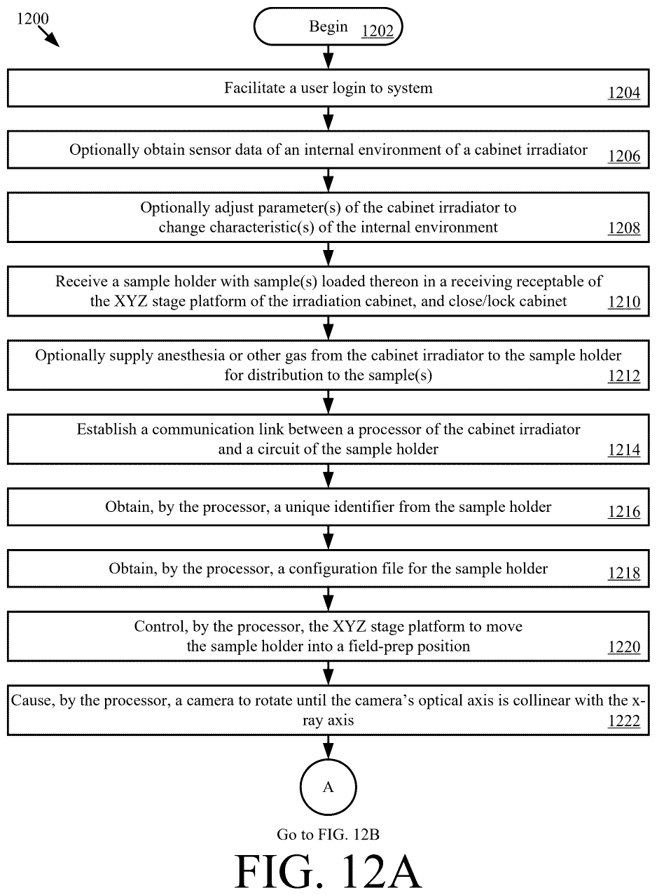

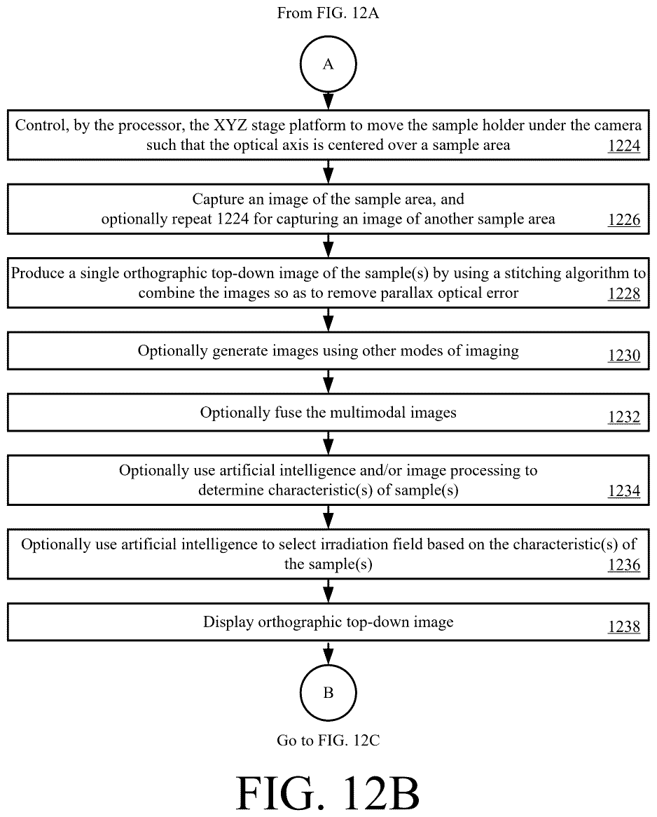

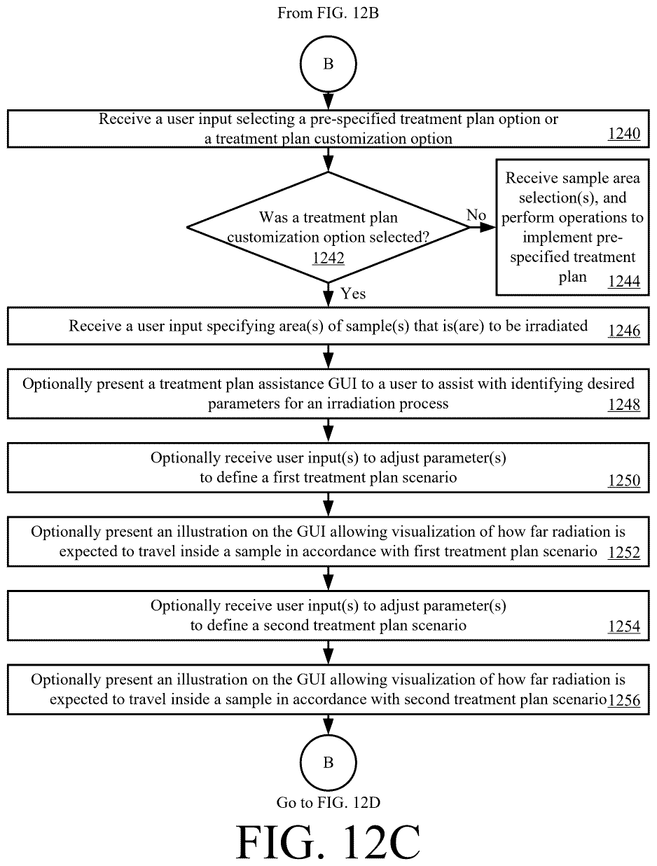

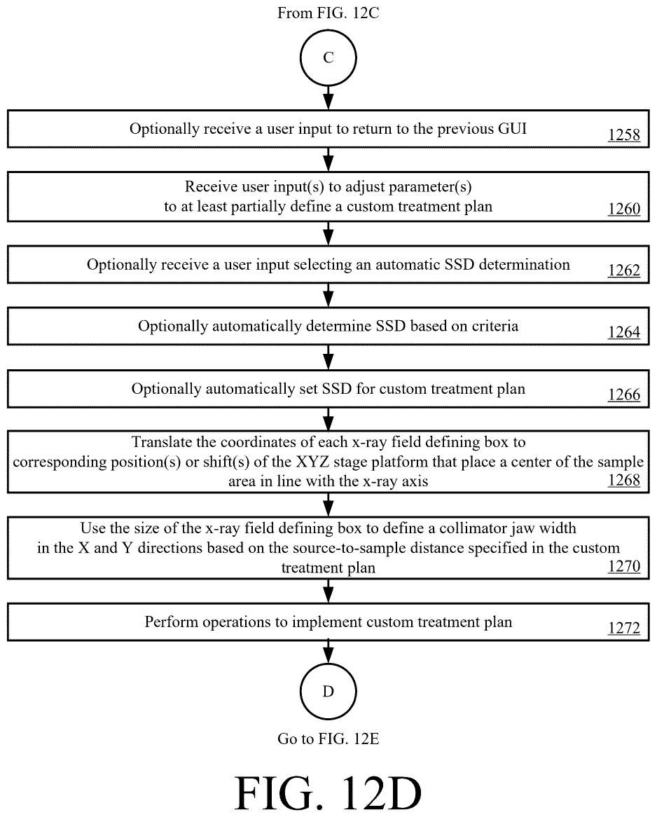

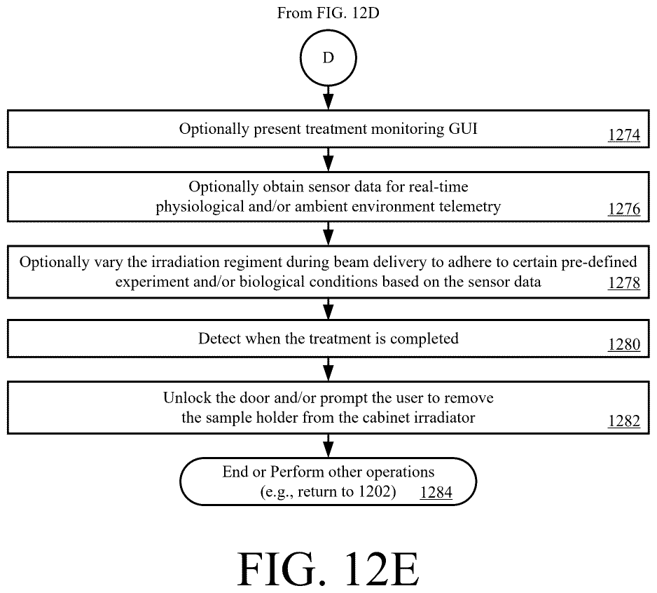

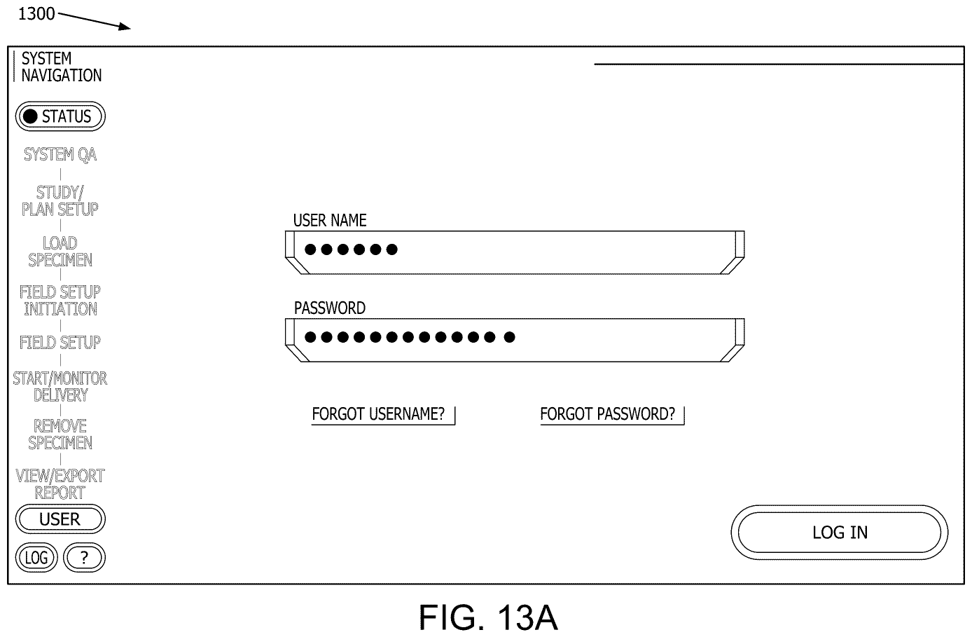

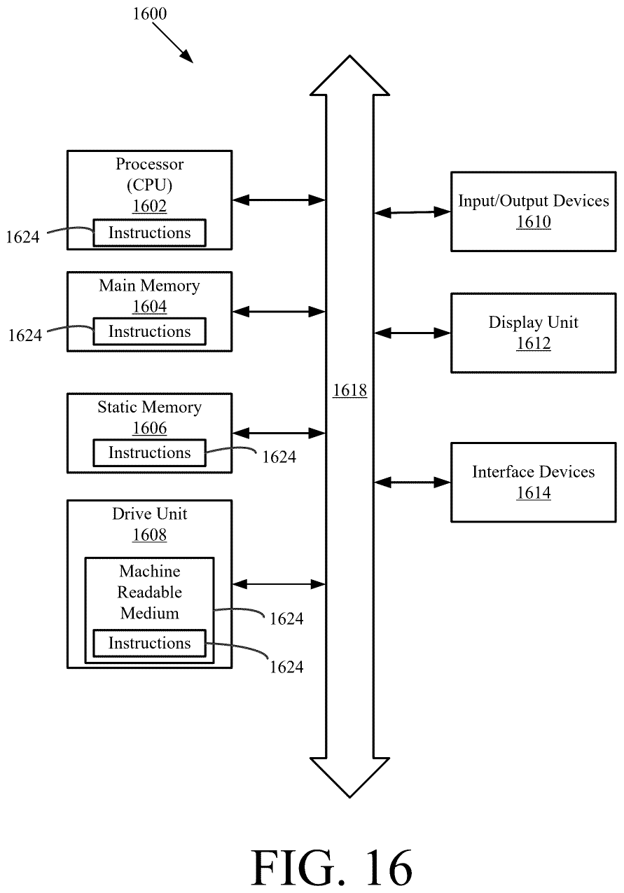

FIG. 12 provides a flow diagram of an illustrative method 1200 for operating the cabinet irradiator 100. Method 1200 may include more or less operations than that shown in FIG. 12. The operations may be performed in the same or different order than that shown. Method 1200 may be performed by a processor (e.g., processor 1602 of FIG. 16 and/or circuit 1130 of FIG. 11), a computing device (e.g., computing device 300 of FIGS. 3 and/or 1600 of FIG. 16), and/or a cabinet irradiator (e.g., cabinet irradiator 100 of FIG. 1). Method 1200 will be discussed below in relation to the processor for ease of illustration. The present solution is not limited in this regard.

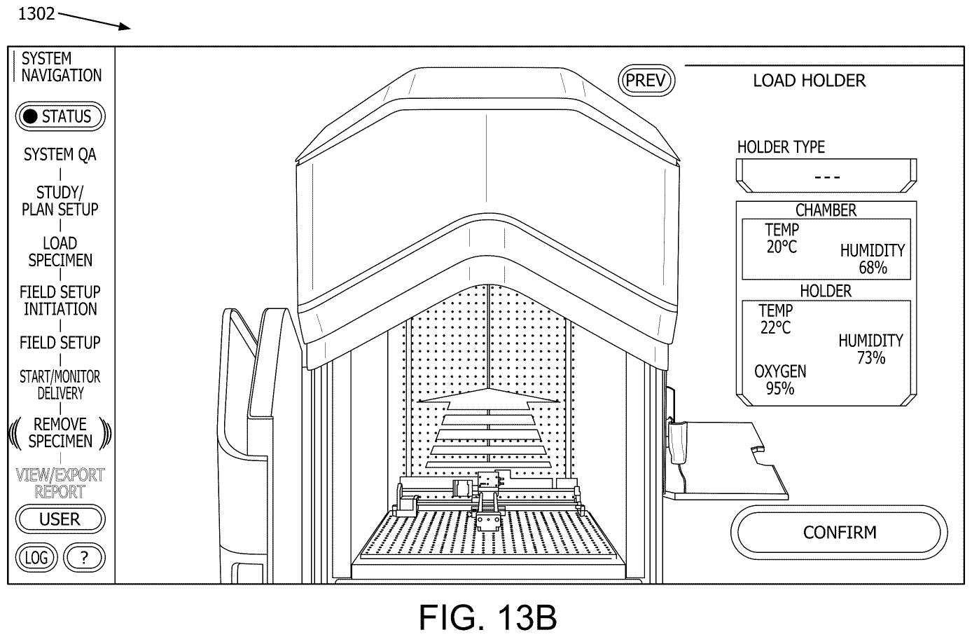

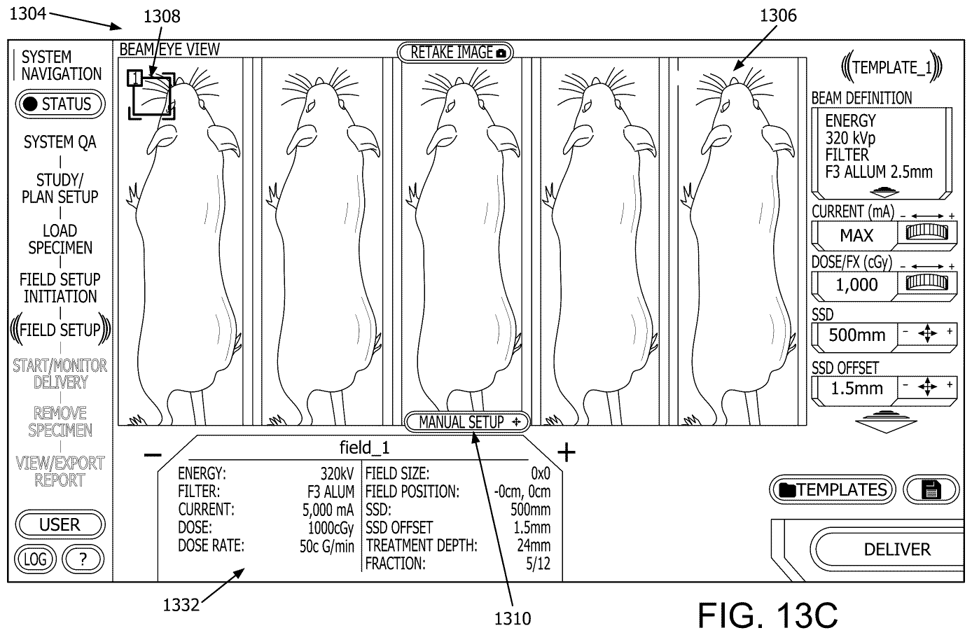

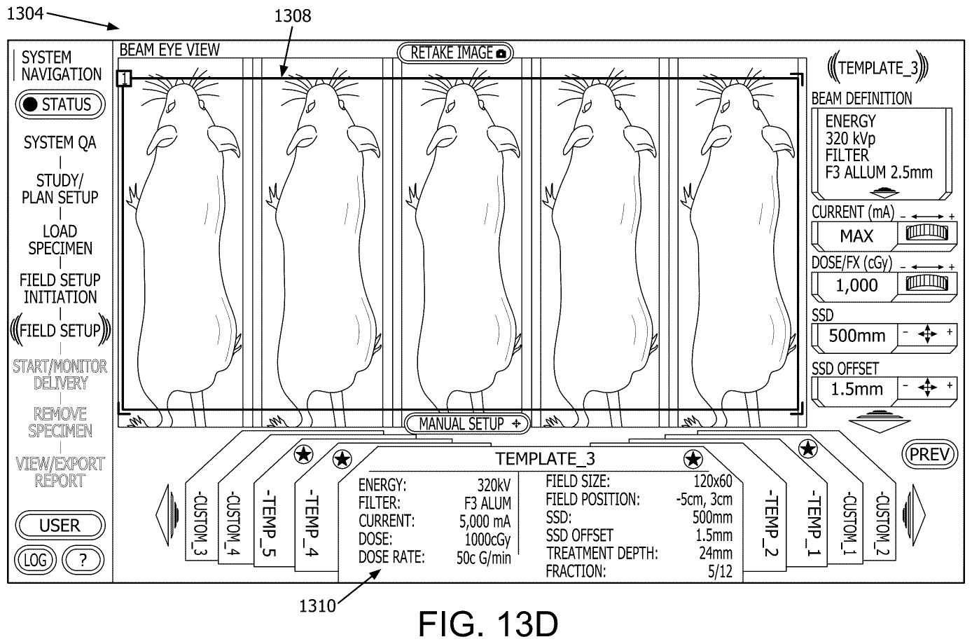

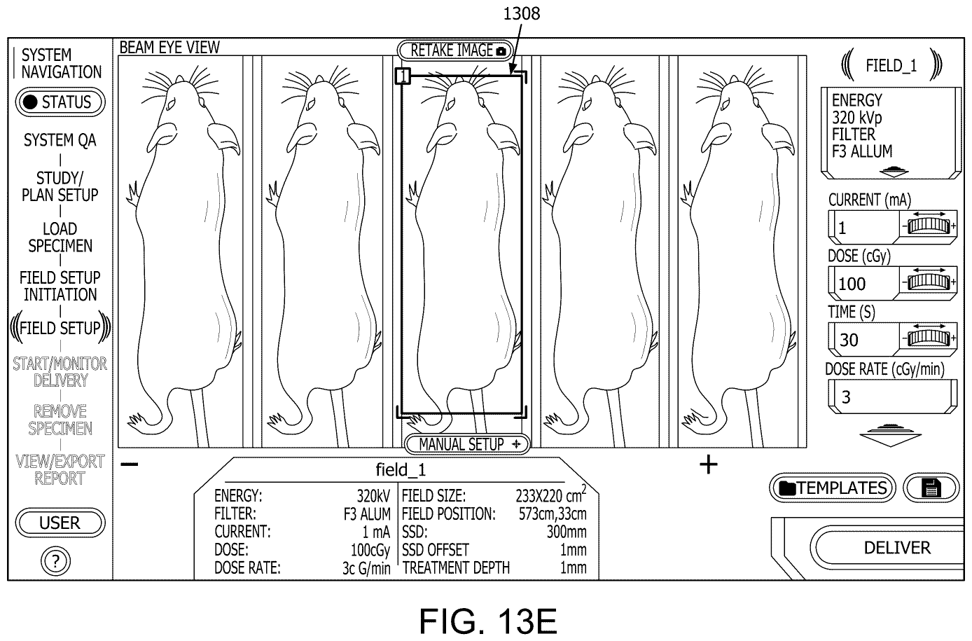

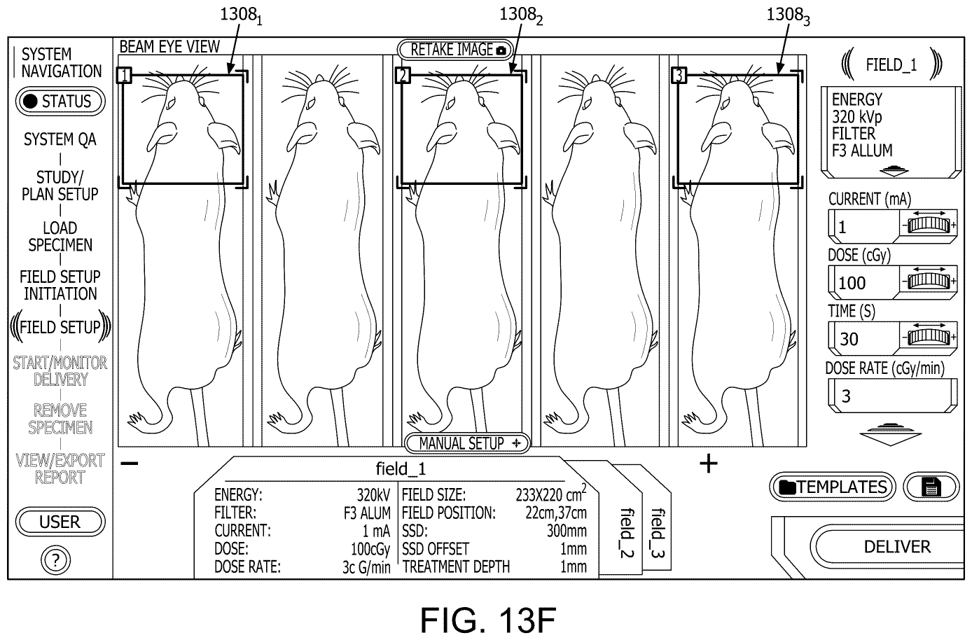

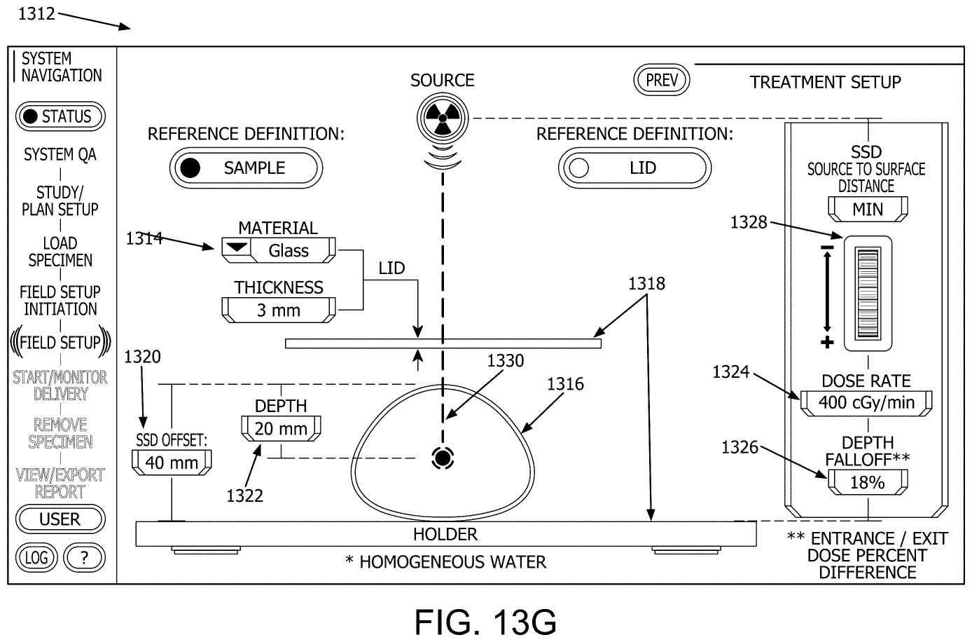

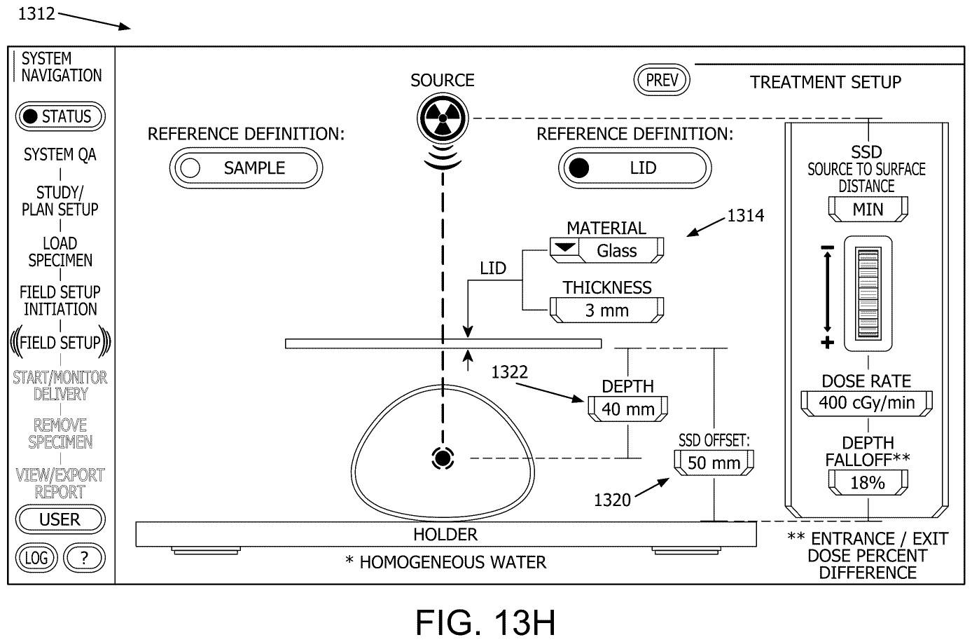

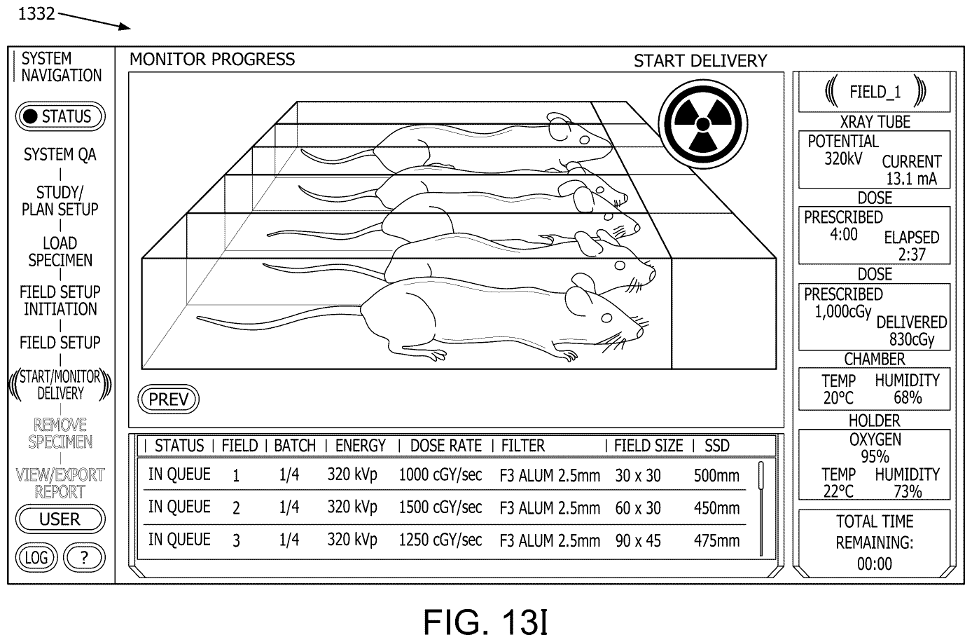

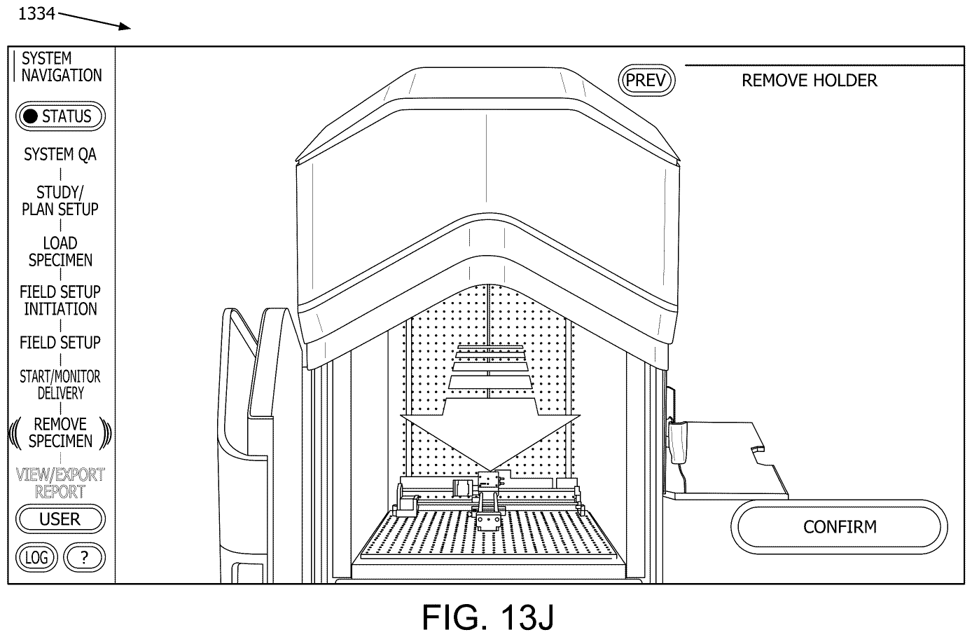

As shown in FIG. 12A, method 1200 begins at block 1202 and continues to block 1204 where the processor facilitates a user login. Any known or to be known user login process can be used herein. An illustrative graphical user interface (GUI) 1300 for user login is provided in FIG. 13A. Upon a successful user login, the processor optionally obtains sensor data of an internal environment of the cabinet irradiator. The sensor data can include, but is not limited to, a temperature, a humidity, and/or an oxygen level. The sensor data may be generated by sensor(s) (e.g., sensors(s) 1020 of FIG. 10) disposed in an irradiation chamber (e.g., irradiation chamber 200 of FIG. 2).

In block 1210, the cabinet irradiator receives a sample holder (e.g., sample holder 200 of FIG. 2) in a receiving receptacle of the XYZ stage platform (e.g., XYZ stage platform 308 of FIG. 3) thereof. The sample holder is loaded with sample(s) (e.g., sample 1102 of FIG. 11). A door (e.g., door 104 of FIG. 1) of the cabinet irradiator may be closed and/or locked in block 1210. An illustrative GUI 1302 is shown in FIG. 12B instructing the user to insert the sample holder into the receiving receptacle of the XYZ stage platform. In some scenarios, the sample holder may be inserted automatically by a robotic arm rather than by the user.

In optional block 1212, the processor may perform operations to cause anesthesia or other gas may be supplied from the cabinet irradiator to the sample holder for distribution to the sample(s). The other gas can include, but is not limited to, oxygen. The operations can include, but are not limited to: controllably and/or selectively opening a valve of a source to allow the gas to flow from the source through a delivery tube of the cabinet irradiator; controllably and/or selectively cause a valve of the sample holder to open to allow the gas to further flow through a gas delivery tube (e.g., gas delivery tube 1128 of FIG. 11) of the sample holder to a gas mask (e.g., gas mask 1112 of FIG. 11). The gas source may be internal to or external to the cabinet irradiator.

In block 1214, a communication link is established between the processor of the cabinet irradiator and a circuit (e.g., circuit 1130 of FIG. 11) of the sample holder. The communication link may be wired or wireless. In the wired scenario, the communication link is facilitated by a connector of the XZY stage platform (e.g., connector 1010 of FIG. 10) and a connector of the sample holder (e.g., connector 1124 of FIG. 11).

In block 1216, the processor obtains a unique identifier from the sample holder. The unique identifier is used by the processor in block 1218 to obtain a configuration file for the sample holder. The configuration file may be obtained from an internal datastore and/or a remote datastore (e.g., a database accessible by a server connected to a network such as the Internet or Intranet). The configuration file can include information specifying geometric dimensions of the sample holder and/or characteristics of the sample holder. The geometric dimensions can include, but are not limited to, length, width, and/or height or thickness. The characteristics can include, but are not limited to, material and/or radiation properties. The sample holder may be formed of glass or plastic.

In block 1220, the processor controls the XYZ stage platform to move the sample holder into a field-prep position. The processor causes a camera (e.g., camera 210 of FIG. 2) to rotate until the camera's optical axis is collinear with the x-ray axis, as shown by block 1222. FIG. 3 shows the camera's optical axis collinear with the x-ray axis. As such, the camera's optical axis and the x-ray axis are referenced in FIG. 3 by number 306. Upon completing the operations of block 1222, method 1200 continues to block 1224 of FIG. 12B.

Block 1224 involves controlling, by the processor. The XYZ stage platform to move the sample holder under the camera such that the optical axis is centered over a sample area of the sample holder. An image is captured of the sample area. A sample holder may comprise multiple adjacent sample areas. As such, multiple images are captured by the camera in block 1226. Each image shows at least one of the sample areas. The processor performs operations in block 1228 to produce a single orthographic top-down image of the sample area(s) by using a stitching algorithm to combine the images in a manner that removes parallax optical error. Any known or to be known stitching algorithm can be used here.

In optional block 1230, other images may be generated using other modes of images. The multimodal images may be fused in block 1232. The other modes of imaging can include, but are not limited to: optical imaging (e.g., providing optical and physiological view of the sample or subject); confocal microscopy imaging (e.g., providing Maximum Intensity Projection (MIP) in-vivo high resolution and deep depth of focus images, and/or providing detailed observation of cellular and viral structures; bioluminescence (BLI) and biofluorescence (BFI) in-vivo imaging (e.g., providing molecular imaging capability with a diverse range of optical reagents, and/or offering single mode 2D optical and 3D tomography molecular in-vivo imaging of cellular samples and phenotype subjects); infrared imaging (e.g., providing real-time infrared (IR) imaging of the subject or cellular sample to trace its physiological and metabolic response to irradiation and/or tested agent/compound); photoacoustic imaging (e.g., providing real-time vascular metabolic response to the experiment during the irradiation sequence of a phenotype subject) and/or computed tomography imaging (e.g., providing pre and post irradiation 3D anatomical computed tomography of the irradiated phenotype subject). At least two or all of the multimodal imaging apparatuses may be fused by the system computer software and/or the treatment planning system (TPS) and integrated in the sample/subject file for irradiation image guidance and post-irradiation sample/subject experiment analysis.

In optional block 1234, the processor uses artificial intelligence (AI) and/or image processing to determine characteristic(s) of the sample(s). For example, the AI may be configured to: identify animal anatomy including (but not limited to) size and/or weight; identify cell plates or petri dishes including (but not limited to) type, size, and/or volume of liquid in each well; and/or identify surface tumors and select the appropriate irradiation field. The AI may be used by the processor in block 1236 to select an irradiation field based on the characteristics of the sample(s).

The AI can use trained machine learning algorithms. Any machine learning algorithm can be used herein without limitation. For example, one or more of the following machine learning algorithms is employed here: supervised learning; unsupervised learning; semi-supervised learning; and reinforcement learning. The learned information by the machine learning algorithm can be used to generate rules for: detecting, identifying and/or classifying animal anatomy, cell plates, petri dishes, and/or surface tumors; and/or selecting an irradiation field from a plurality of possible irradiation fields.

The orthographic top-down image is displayed in block 1238. An illustrative GUI 1304 is shown in FIG. 13C that includes a displayed orthographic top-down image 1306. The image 1306 shows that five samples are loaded onto the sample holder. The present solution is not limited in this regard. Any number of samples may be loaded on the sample holder. Upon completing the operations of block 1238, method 1200 continues to block 1240 of FIG. 12C.

Block 1240 involves receiving, by the processor, a user input selecting a pre-specified treatment plan option or a treatment plan customization option. An illustrative GUI 1304 is shown in FIG. 13C with a widget 1310 configured to facilitate the user selection of a treatment plan option. The widget 1310 is shown as comprising a virtual button. The present solution is not limited in this regard. Widget 1310 can include any other type of widget for selecting one or more options.

If the pre-specified treatment plan option is selected [1242: NO], then method 1200 continues to block 1244 where a sample area selection(s) is(are) made. Each sample area selection is facilitated by a movable (e.g., draggable) and resizable (e.g., expandable) virtual box overlaid on the displayed image. An illustrative GUI 1304 showing such a virtual box 1308 is provided in FIG. 13C. Box 1308 represents an augmented reality representation of the x-ray field and allows the user to visualize the extent of the radiation beam. Box 1308 may be dragged or otherwise moved to any location within image 1306 and may be expanded and/or collapsed to change its overall size. The predefined holder boundaries stored in the system may limit the selectable field regions or locations within the image to which the box may be moved. FIG. 13D shows box 1308 in an expanded state such that it encompasses a majority of the samples. FIG. 13E shows box 1308 in a dragged and expanded state such that it encompasses a majority of a particular one of the five samples. FIG. 13F shows the scenario is which multiple virtual boxes 13081, 13082, 13083 are overlaid on the displayed image. Each box 13081, 13082, 13083 can be moved to different positions relative to the display image and/or resized. In this way, multiple sample areas may be selected.

Operations are also performed in block 1244 by the cabinet irradiator to implement a pre-specified irradiation treatment plan. An illustrative template 1310 for an irradiation treatment plan is shown in GUI 1304 of FIG. 13D. The irradiation treatment plan is defined by a plurality of parameters. The parameters can include, but are not limited to, a beam definition, a current, a dose, a dose rate, a field size, a field position, an SSD, an SSD offset, a treatment depth, a fraction, and/or a time. The beam definition may be defined by an energy parameter and a filter parameter.

If the treatment plan customization option is selected [1242: YES], then method 1200 continues to block 1246 where a user input is received specifying area(s) of sample(s) that is(are) to be irradiated. The area(s) may be selected, for example, using box(es) 1308, 13081, 13082, 13083 shown in FIGS. 13C-13F.

Next in optional block 1248, a treatment plan assistance GUI may be presented to assist with identifying desired parameters for an irradiation process. An illustrative treatment plan assistance GUI 1312 is shown FIG. 13G. GUI 1312 comprises virtual objects showing values for various irradiation treatment plan parameters. Virtual object 1320 shows an SSD offset. Virtual object 1322 shows a depth. Virtual object 1324 shows a dose rate. Virtual object 1326 shows a depth fallout. Virtual object 1328 shows an SSD. Virtual object 1328 may include a widget to allow a user to adjust an SSD. The widget can include, but is not limited to, a slider and/or a wheel widget. GUI 1312 also comprises an illustration showing a cross-sectional view 1316 of a sample, a cross-sectional view 1318 of a sample holder, and visual representation 1330 of how far into the sample the x-rays are expected to penetrate the sample given the sample holders dimensions/characteristics and/or the parameter settings for dose rate and SSD. Virtual objects 1314 show the geometric dimension(s) and/or characteristic(s) of the sample holder. The location of virtual objects 1314, 1320, 1322 may vary while the GUI 1312 is being displayed as shown by FIGS. 13G-13H. Changes to the location(s) of the virtual objects 1314, 1320, 1322 may be responsive to and/or based on an adjustment to the SSD. The ability to adjust the SSD allows a user to define different irradiation treatment plan scenarios and visualize the x-ray beam penetration into the sample. The user can then identify parameter settings for a customized irradiation treatment plan.

In this regard, method 1200 may include operations of optional blocks 1250-1256. These operations involve: receiving user input(s) to adjust parameter(s) to define a first irradiation treatment plan scenario; presenting an illustration on the GUI allowing visualization of how far radiation is expected to travel inside a sample in accordance with first irradiation treatment plan scenario; receiving user input(s) to adjust parameter(s) to define a second irradiation treatment plan scenario; and presenting an illustration on the GUI allowing visualization of how far radiation is expected to travel inside a sample in accordance with second irradiation treatment plan scenario. FIGS. 13G-13H provide illustrations that are useful for understanding these operations. Thereafter, method 1200 continues to block 1258 of FIG. 12D.

Block 1258 involves optionally receiving by the processor a user input to return to a previous GUI (e.g., GUI 1304 of FIG. 13C). This GUI includes a means to allow a user to define parameter value(s) for a custom irradiation treatment plan. As such, the processor may receive user input(s) in block 1260 for adjusting parameter value(s) to at least partially define the custom irradiation treatment plan. FIG. 13C shows a tab with text boxes in GUI 1304. The text boxes allow a user to change the values for one or more of the following parameters-energy, filter, current, dose, dose rate, field size, field position, SSD, SSD offset, treatment depth, and/or fraction. The present solution is not limited in this regard. Other parameters may additionally or alternatively be modifiable. For example, as shown in FIG. 13E, the modifiable parameters include time in addition to energy, dose, and/or dose rate.

In some scenarios, the cabinet irradiator may be provided with an automatic SSD determination feature. Accordingly, method 1200 may include operations of optional blocks 1262-1266. These operations involve: receiving a user input selecting an automatic SSD determination; automatically determining SSD based on criteria; and/or automatically setting SSD for custom irradiation treatment plan. Known parameters of the desired field and inherent system x-ray field may be used to automatically define the SSD based on identified criteria. One example of an identified criteria may be to always maximize the dose rate based on a chosen field size. The output x-ray field of the x-ray tube is a cone of half angle θ whose apex represents the x-ray source within the x-ray tube. A certain minimum SSD is defined as the closest distance reasonably achievable from the sample holder to the x-ray source, which may be limited by intermediate components such as the filter wheel (e.g., filter wheel 208 of FIG. 2) and collimator (e.g., collimator 206 of FIG. 2). The maximum allowed SSD is defined as the furthest distance a sample may reach from the source limited by the irradiation chamber interior dimensions and XYZ stage travel. Due to the typical sample geometry of small animals or cell holding plates, the usable irradiation field is typically a square or rectangle inscribed within the circle defined by the intersection of the irradiation field cone and a plane at a distance from the x-ray source defined by the source-to-surface distance. In the event the desired field size is smaller than the inscribed quadrilateral at the minimum SSD, the SSD may be automatically set to the minimum SSD. In the event the desired field sized is larger than the inscribed quadrilateral at the minimum SSD, the SSD may be automatically set to be shortest distance to the source such that desired field size is still capable of being inscribed in a circle at the selected SSD. In such a way, the SSD is automatically set via the Z axis of the XYZ stages.

In block 1268, the processor performs operations to translate the coordinates of each x-ray field defining box (e.g., box(es) 1308, 13081, 13082, 13083 of FIGS. 13C-13F) to corresponding position(s) or shift(s) of the XYZ stage platform that place a center of the sample area in line with the x-ray axis. For example, the processor uses the x-ray field defining box centroid to define the necessary shift of the XY stages needed to place the desired sample area in line with the x-ray axis. The shift needed for the stages to move the sample area to align with the specified field centroid (desired field centroid coordinate). For example, if the desired field XY centroid is located at (−1 cm, 2 cm), then the stage translation is 1 cm in the positive X direction and 2 cm in the negative Y direction. The present solution is not limited to the particulars of this example. The translation may also be facilitated using: the grid of apertures formed in the sample holder; and/or a positional relationship between each x-ray defining box as shown in the display image. In the grid scenario, the process may determine, for example, the shifts needed for the stages to move the center aperture of the grid into alignment with the specified field centroid.

In block 1270, the processor uses the size of the x-ray field defining box to define a collimator jaw width in the X and Y directions based on the SSD specified in the custom irradiation treatment plan. The collimator opening width W=(Desired Field Size)*(Source to Collimator Distance)/(SSD). The Source to Collimator Distance is distance from the x-ray source in the x-ray tube to the plane defining the collimator jaws of interest where the plane is perpendicular to the beam axis.

In block 1272, the processor performs operations to cause the cabinet irradiator to implement the custom irradiation treatment plan. The cabinet irradiator is configured with high precision beam forming. Any known or to be known beam forming technique can be used here. The cabinet irradiation may comprise a beam forming/shaping X-ray source for shaping the irradiating beam into high resolution conformal beamlets, which will allow for a precise irradiation of specific areas of the subject or sample. The beam forming mechanism and method allows for delivering multiple differentiated beamlets with varying doses and irradiated surfaces/shapes. This mechanism and method function in conjunction, or separately of the physical beam collimating apparatus.

Method 1200 then continues to block 1274 of FIG. 12E. Block 1274 involves optionally presenting a treatment monitoring GUI. An illustrative treatment monitoring GUI 1332 is shown in FIG. 13I. GUI 1332 may be configured to present video of the sample(s) during the irradiation process as well as present measured characteristics of the internal environment of the cabinet irradiator and/or characteristics of the sample holder. For example, the current temperature, humidity and oxygen level of the irradiation chamber are presented in GUI 1332. The current temperature, humidity and oxygen level of the sample holder are also presented in GUI 1332. Accordingly, method 1200 may also involve obtaining sensor data for real-time physiological and/or ambient environment telemetry as shown by block 1276. Other information may also be presented in GUI 1332 such as the x-ray tube potential, x-ray tube current, prescribed time, elapsed time, remaining time, prescribed dose, delivered dose.

In optional block 1278, the irradiation regiment may be varied during beam delivery to adhere to certain pre-defined experiment and/or biological conditions based on the sensor data obtained in block 1276. This feature of the cabinet irradiator may be referred to as Real-Time Subject/Sample Irradiation Augmentation. The cabinet irradiation may comprise physiological and chamber ambience sensors that provide the system with real-time physiological and ambient environment telemetry, which will vary the irradiation regiment during beam delivery to adhere to certain pre-defined experiment and biological conditions. This mechanism may be governed by a combination of design of experiment algorithm and an AI neuro network, which will optimize the study and experiment outcome and results.

In block 1280, the processor detects when the treatment is completed. The cabinet irradiator's door may then be unlocked and/or opened as shown by block 1282. A GUI may be presented with instructions to remove the sample holder from the cabinet irradiator. An illustrative GUI 1334 is shown in FIG. 13J with an arrow for instructing the removal of the sample holder from the cabinet irradiator. The sample holder may be removed by the user or by a robotic arm. Subsequently, method 1200 continues to block 1284 where it ends or other operations are performed. The other operations can include, but are not limited to, returning to block 1202 of FIG. 12A.

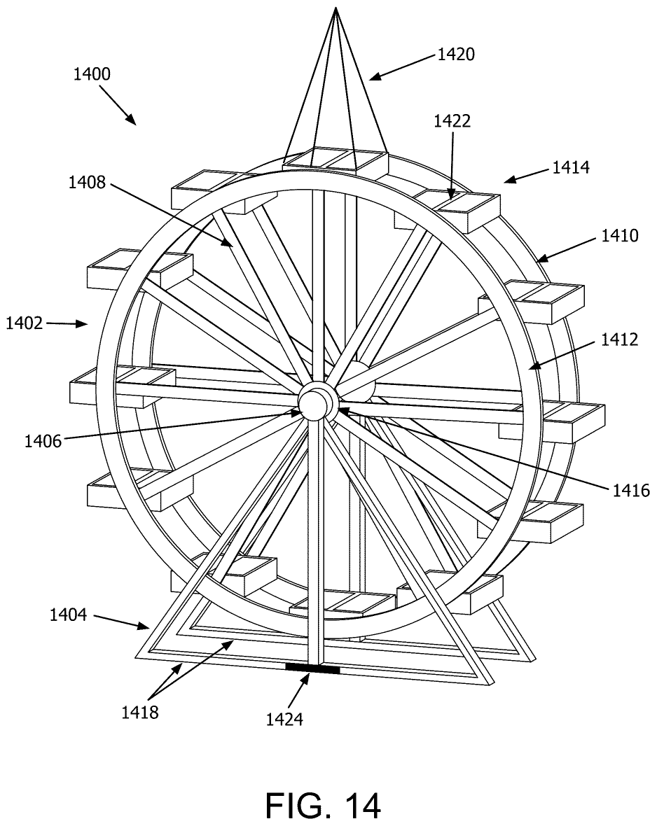

FIG. 14 provides a perspective view of a carousel 1400 for samples (e.g., sample(s) 1102 of FIG. 11). Carousel 1400 comprises a vertical revolving wheel 1402 with sample holders 1414 suspended on its outer edge. Vertical revolving wheel 1402 is sized, shaped and/or otherwise designed to be inserted into the irradiation chamber 200. The sample holder(s) 1414 may be the same as, similar to, or different than sample holder 202. Thus, the above discussion of sample holder 202 is sufficient for understanding sample holder(s) 1414. However, it should be noted that connector 1124 would mate with a connector of wheel 1402 rather than a connector of the cabinet irradiator. Also, sample holder 1414 may comprise multiple protruding sidewalls rather than a single protruding side wall. This multiple sidewall configuration facilitates retention of sample(s) on the sample holder while the wheel 1402 rotates or is moved linearly by an XYZ stage platform.

Wheel 1402 is structurally supported by stand 1404 in its vertical position. The sample holders 1414 are suspended between rims 1410, 1412 of wheel 1402. Each sample holder 1414 is configured to rotate relative to the rims 1410, 1412 such that the sample holder remains horizontally aligned with the ground or support structure on which the carousel 1400 sits. This ensures that any sample(s) loaded on the sample holder 1414 will not fall off while the carousel 1400 is inside the irradiation chamber 200. X-ray beam 1420 is applied to the sample on the top-most sample holder at the given time.

The sample holders may be selectively moved in and out of alignment with the x-ray axis via rotation of the wheel 1402 about axel 1406. Spokes 1408 are provided to couple the rims 1410, 1412 to the hub 1416. Hub 1416 is coupled to axel 1406 to facilitate rotation of the wheel 1402.

Carousel 1400 may be used when there is a relatively large batch of samples that are to be irradiated during an irradiation process. Carousel 1400 is designed to allow for large batches of samples to be irradiated such that there is maximum dose rate. Such an application requires a minimal distance of the sample to the x-ray source. At this minimal distance, the extent of the usable field size is small due the output radiation cone of the x-ray tube. The limiting field size limits the number of simultaneous samples that may be irradiated in a given session. Carousel 1400 is designed as a vertically oriented cylindrical carousel with the axis of the cylinder being parallel to the irradiation chamber floor. However, the present solution is not limited to this vertically oriented cylindrical carousel architecture. Horizontally oriented cylindrical carousel architecture may be employed in some scenarios such that the axis of cylinder is perpendicular to the irradiation chamber floor.

During use, a large batch of samples may be placed on the carousel 1400 such that a small number of samples is rotated into the irradiation field, irradiated, and then automatically moved out of the irradiation area after the desired dose has been reached upon which the next set of samples out of the batch is rotated into place. In order to reduce secondary scatter, shielding plates 1422 may be present between the samples within a single sample set holder and under the sample irradiation volume to reduce over irradiation of downstream samples.

Carousel 1400 may comprise sensor(s) and/or other electronic device(s) configured to monitor and measure real-time dose for additional dosimetric verification. In this regard, planar member(s) 1418 may be configured to comprise a connector 1424 that may be plugged into a connector of the XYZ stage platform. Connector 1424 may be similar to connector 1124 of sample holder 202 such that a communications link between the carousel 1400 and the processor of the cabinet irradiator may be established and/or such that a gas may be supplied to the carousel 1400 from the cabinet irradiator. Gas delivery tubes may be disposed within and/or coupled to stand 1404, hub 1416, spokes 1408, and/or rims 1410, 1412. Additionally or alternatively, one or more of the listed components 1404, 1408, 1410, 1412, 1416 may comprise gas delivery tube(s). In this way, the listed component(s) 1404, 1408, 1410, 1412, 1416 would have multiple purposes. For example, stand 1404 may have the following purposes: (i) structurally support wheel 1402 in a vertical or horizontal revolving position; (ii) facilitate an electrical connection between sensor(s) of each sample holder 1414 and a processor of the cabinet irradiator; and/or (iii) facilitate the supply of gas from a gas source to each sample holder. Each of the other listed components 1408, 1410, 1412, 1416 may also have purposes (ii) and (iii) in addition to the known purpose of a hub, a spoke or a rim.

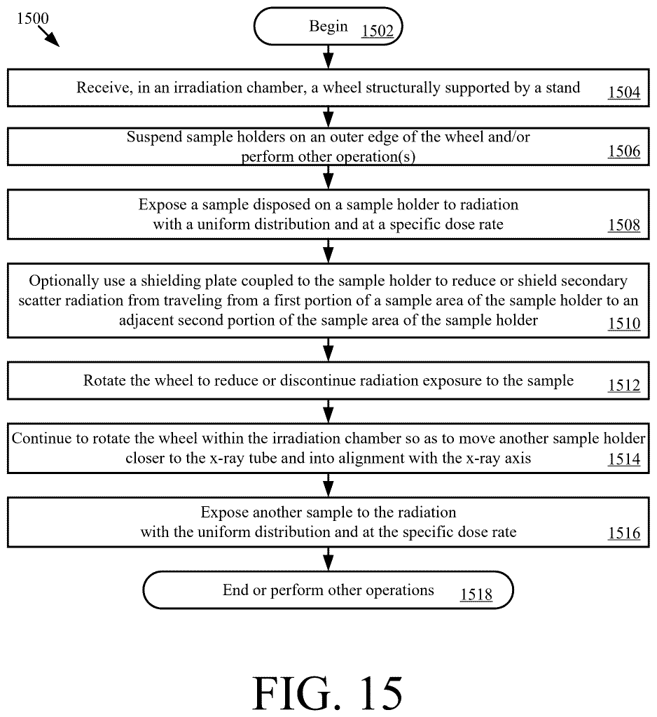

FIG. 15 provides a flow diagram of an illustrative method 1500 for operating an irradiation system. The operations of method 1500 may be performed in the same order as that shown or in a different order than that shown. For example, the operations of block 1506 may occur prior to the operations of block 1504.

Method 1500 begins at block 1502 and continues to block 1504 where a wheel (e.g., carousel 1400 is received in an irradiation chamber (e.g., irradiation chamber 200 of FIG. 2) of an irradiator (e.g., cabinet irradiator 100 of FIG. 1). As shown by block 1506, sample holders (e.g., sample holders 202 of FIGS. 2 and/or 1414 of FIG. 14) are suspended on an outer edge of the wheel in a manner that maintains a horizontal alignment of each of the sample holders with a surface of the irradiation chamber on which the stand sits. The wheel may comprise a first circular rim and a second circular rim parallel to the first circular rim. An axis of a cylinder defined by the first and second rims may extend parallel to or perpendicular to an irradiation chamber floor.

A surface-to-skin distance parameter for an irradiation treatment plan may be defined by a distance between the x-ray source of the irradiator and a sample disposed in a top most one of the sample holders. As such, block 1506 may also optionally involve modifying a value of the surface-to-skin distance parameter by adjusting a height of a portion of a sample area of the first sample holder. Additionally or alternatively, block 1506 may also optionally involve: using a restraining member coupled to the first sample holder to restrain the first sample in a sample area of the first sample holder; supplying power to the circuit (e.g., circuit 1130 of FIG. 11) of the sample holder via an electrical connection between the sample holder and the wheel and via an electrical connection between the wheel and a circuit (e.g., computing device 300 of FIG. 3) of the irradiator; performing operations by the circuit of the sample holder to detect characteristics of an internal environment of the irradiator; connecting a gas delivery tube of the irradiator to a gas delivery tube of the first sample holder via the wheel; receiving at the first sample holder gas from the irradiator; using a valve of the first sample holder to control a flow of gas to a sample area of the first sample holder; using a gas mask coupled to the first sample holder to direct and retain at least some of the gas within the sample area of the first sample holder.

Next in block 1508, a sample (e.g., sample 1102 of FIG. 11) is exposed to radiation with a uniform distribution and at a specific dose rate. The sample is disposed on the sample holder that is the closest to an x-ray tube (e.g., x-ray tube 204 of FIG. 2) of the irradiator. Block 1510 involves optionally using shielding plate(s) (e.g., shielding plate 1110 of FIG. 11) coupled to the sample holder to reduce or shield secondary scatter radiation from traveling from a first portion of a sample area of the sample holder (e.g., an area one side of the shield) to an adjacent second portion of the sample area of the sample holder (e.g., an area on another side of the shield).

The wheel is rotated in block 1512 to reduce or discontinue radiation exposure to the sample. Rotation of the wheel within the irradiation chamber causes the sample holder to move away from the x-ray tube (e.g., x-ray tube 204 of FIG. 2) and out of alignment with an x-ray axis (e.g., x-ray axis 306 of FIG. 3). Rotation of the wheel is continued in block 1514 within the irradiation chamber so as to move another sample holder closer to the x-ray tube and into alignment with the x-ray axis. A grid pattern of apertures of the sample holder may be used to facilitate a translation of virtual box coordinates in a graphical user interface of the irradiator to shifts of an XYZ stage platform of the irradiator. As such, block 1514 may also optionally involve using the shifts of an XYZ stage platform to align a sample (disposed on the another sample holder) with the x-ray axis. This sample is exposed to radiation in block 1516. This sample is disposed on the sample holder which was moved closer to the x-ray tube. Subsequently, method 1500 continues to block 1518 where it ends or other operations are performed.

The above-described carousel 1400 and/or method 1500 may be used in various applications. The applications include, but are not limited to, bone marrow ablation applications, flash radiation therapy modeling in preclinical oncology, cellular radiation experiments, applications in which a relatively large number of samples should be analyzed and/or passed through a beam, and/or other applications in which provision of radiation with a uniform distribution and a relatively high dose rate is desired, needed or required. Bone marrow ablation in mice enables rapid and uniform total body irradiation across large mouse cohorts prior to stem cell transplantation. Flash radiation therapy modeling in preclinical oncology supports studies that require repeated, reproducible whole-body or tumor-targeted FLASH irradiation. FLASH can often be achieved by minimizing SSD usually at the expense of batch size. Cellular radiation experiments allow large batches of cell cultures in flasks or multi-well plates to be irradiated at matched dose levels particularly when high dose rates are desirable to analyze cellular to dosage times.

The sample wheel of carousel 1400 introduces a novel mechanical and functional architecture that significantly enhances the utility of cabinet irradiators for high-dose, high-throughput applications. Unlike conventional static sample holders or simple rotating platforms, the Ferris wheel-like design of the irradiation system allows for multiple samples to be irradiated sequentially while maintaining the short SSD necessary for high dose rate delivery. By rotating samples individually into the beam path while keeping each tray rigid and perpendicular to the s-ray beam, the device ensures uniform, repeatable dosing across all samples—an important feature for experimental reproducibility.

Another important innovation is the use of an integrated shielding system that isolates non-target trays from scatter or leakage radiation, a limitation in many current batch irradiation designs. The mechanical system for tray rotation and alignment is designed for precision, providing reliable positioning and eliminating manual variability. This capability makes the sample wheel of carousel 1400 particularly well-suited for protocols requiring strict control of dose delivery across many replicates, including those used in regulatory or preclinical environments.

The system is designed for compatibility with known or to be known irradiators, requiring minimal modification or adaptation. Its plug-and-play modularity and automation-ready interface make it a highly adaptable and scalable solution. Additionally, the system's modular design allows easy cleaning, tray replacement, and repeat use, supporting high-throughput experimental demands while maintaining rigorous standards for reproducibility and radiation safety. These features represent a significant advancement over existing methods of delivering high dose radiation to multiple samples in constrained spaces.

In various use cases, the following procedure may be following using the sample wheel. A first step relates to the positioning of the device. The device is positioned by: placing the wheel system in a designated, calibrated position inside the cabinet irradiator (e.g., cabinet irradiator 100 of FIG. 1) such that the beam is aligned with the irradiation window; and connecting any power, control and/or data cables between the sample wheel system and the controller or irradiator system. A second step relates to sample preparation and loading. In this regard, biological or material samples may be prepared in the modular, removable sample trays or holders designed for precise and reproducible positioning. The sample trays or holders are loaded into wheel positions. The design may ensure that each sample tray or holder maintains perpendicular alignment with the incident x-ray beam. A third step relates to configuration and start. The third step may involve: inputting desired radiation dose parameters into the irradiator control interface; and beginning the automated irradiation cycle by pressing a Start button (physical or virtual). A fourth step relates to the automated irradiation process. During the automated irradiation process, the irradiator emits radiation to the first sample tray or holder at a short SSD to achieve a high dose rate. Once the desired dose is delivered, radiation stops. The sample wheel mechanism automatically rotates the next sample tray or holder into position, preserving precise alignment and shielding all non-targeted trays. The process repeats until all trays have been sequentially irradiated. A fifth step involves sample retrieval. Once the full irradiation cycle is complete, the irradiated samples are unloaded for further processing or analysis.

Referring now to FIG. 16, there is shown an illustrative architecture for a computing device 1600. The camera 210 of FIG. 2 and/or computing device 300 of FIG. 3 is/are the same as or similar to computing device 1600. As such, the discussion of computing device 1600 is sufficient for understanding the components 210 of FIG. 2 and/or computing device 300 of FIG. 3.

Computing device 1600 may include more or less components than those shown in FIG. 16. However, the components shown are sufficient to disclose an illustrative solution implementing the present solution. The hardware architecture of FIG. 16 represents one implementation of a representative computing device configured to receive information, process the receive information, transmit information and/or control operations of one or more robots, as described herein. As such, the computing device 1600 of FIG. 16 implements at least a portion of the method(s) described herein.

Some or all components of the computing device 1600 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 16, the computing device 1600 comprises a user interface 1602, a Central Processing Unit (CPU) 1606, a system bus 1610, a memory 1612 connected to and accessible by other portions of computing device 1600 through system bus 1610, a system interface 1660, and hardware entities 1614 connected to system bus 1610. The user interface can include input devices and output devices, which facilitate user-software interactions for controlling operations of the computing device 1600. The input devices include, but are not limited to, a physical and/or touch keyboard 1650. The input devices can be connected to the computing device 1600 via a wired or wireless connection (e.g., a Bluetooth® connection). The output devices include, but are not limited to, a speaker 1652, a display 1654, and/or light emitting diodes 1656. System interface 1660 is configured to facilitate wired or wireless communications to and from external devices (e.g., network nodes such as access points, etc.).

At least some of the hardware entities 1614 perform actions involving access to and use of memory 1612, which can be a Random Access Memory (RAM), a disk drive, flash memory, a universal serial bus (USB) drive and/or another hardware device that is capable of storing instructions and data. Hardware entities 1614 can include a disk drive unit 1616 comprising a computer-readable storage medium 1618 on which is stored one or more sets of instructions 1620 (e.g., software code) configured to implement one or more of the methodologies, procedures, or functions described herein. The instructions 1620 can also reside, completely or at least partially, within the memory 1612 and/or within the CPU 1606 during execution thereof by the computing device 1600. Memory 1612 and CPU 1606 also can constitute machine-readable media. The term “machine-readable media”, as used here, refers to a single medium or multiple media (e.g., a centralized or distributed database, and/or associated caches and servers) that store one or more sets of instructions 1620. The term “machine-readable media”, as used here, also refers to any medium that is capable of storing, encoding or carrying a set of instructions 1620 for execution by the computing device 1600 and that cause the computing device 1600 to perform any one or more of the methodologies of the present disclosure.

In view of the forgoing, the present solution concerns implementing systems and methods for operating a cabinet irradiator (e.g., cabinet irradiator 100 of FIG. 1). The methods comprise: receiving a sample holder (e.g., sample holder 202 of FIG. 2, and/or sample holder 1414 of FIG. 14) in a receptacle of an XYZ stage platform (e.g., XYZ platform 308 of FIG. 3) of the cabinet irradiator. Sample(s) (e.g., sample 1102 of FIG. 11A) is(are) disposed on the sample holder; controlling, by a processor (e.g., computing device 300 of FIG. 3 and/or processor 1602 of FIG. 16), the XYZ stage platform to move the sample holder under a camera (e.g., camera 210 of FIG. 2) such that an optical axis (e.g., axis 306 of FIG. 3) is centered over a sample area (e.g., sample area 1136 of FIG. 11A) of the sample holder; displaying, by the cabinet irradiator, a top-down image (e.g., image 1306 of FIG. 13C) of the at least one sample in a first graphical user interface (e.g., GUI 1304 of FIG. 13C) along with at least one virtual box (e.g., virtual box 1308 of FIG. 13C) having an adjustable size and being movable relative to the top-down image; receiving, by the processor, a user input specifying at least one area of the at least one sample that is to be irradiated via movement and or resizing of the at least one virtual box; receiving, by the processor, a user input selecting or defining an irradiation treatment plan; and irradiating, by the cabinet irradiator, the at least one sample in accordance with the irradiation treatment plan.

In some scenarios, the sample(s) may be living. The method further comprises supplying gas from the cabinet irradiator to the sample holder for distribution to the sample(s). The gas can include, but is not limited to, anesthesia or oxygen. The sample holder may comprise a gas distribution tube connected to a gas distribution tube of the cabinet irradiator via a gas port (e.g., gas port 1022 of FIG. 10) of the XYZ stage platform.

In those or other scenarios, the irradiation treatment plan may be selected from a plurality of pre-defined irradiation treatment plans with different values for at least one of a beam definition, a dose, and a source-to-skin distance. The irradiation treatment plan may comprise a custom or customized irradiation treatment plan.

In those or other scenarios, the method also comprises: providing a widget on the first or another graphical user interface that is configured to allow adjustment of one or more treatment plan parameters; and/or receiving another user input adjusting the one or more treatment plan parameters. The treatment plan parameters can include, but are not limited to, a beam definition, a dose, and/or a source-to-skin distance.

In those or other scenarios, the method comprises: displaying a second graphical user interface (e.g., GUI 1312 of FIG. 13G) configured to assist a user with identification of desired parameters for the custom or customized irradiation treatment plan; receiving another user input adjusting the at least one treatment plan parameter to define a first irradiation treatment plan scenario; and/or presenting an illustration in the second graphical user interface that shows a visual representation of how far radiation is expected to travel inside the at least one sample in the first irradiation treatment plan scenario. The second graphical user interface can include, but is not limited to, widget(s) to facilitate adjustment of at least one treatment plan parameter to selectively define different irradiation treatment plan scenarios. The treatment plan parameter(s) can include, but is(are) not limited to, a source-to-skin distance.

In those or other scenarios, the method comprises: receiving another user input adjusting the at least one treatment plan parameter to define a different second irradiation treatment plan scenario; dynamically modifying or replacing the illustration in the second graphical user interface to show a visual representation of how far radiation is expected to travel inside the at least one sample in the different second irradiation treatment plan scenario; and/or receiving another user input specifying the desired parameters for the custom or customized irradiation treatment plan.

In those or other scenarios, the method comprises: automatically determining, by the processor, a source-to-skin distance for the irradiation treatment plan based on a desired field size, a minimum surface-to-skin distance limited by a filter wheel and a collimator of the cabinet irradiator, and a maximum allowed surface-to-skin distance limited by dimensions of an irradiation chamber and XYZ stage travel; setting the source-to-skin distance to the minimum surface-to-skin distance when the desired field width is smaller than a maximum field width; and/or setting the source-to-skin distance to (SSDₘᵢₙ*tan(θ_{Collimator}), when the desired field width is larger than a maximum field width.

In those or other scenarios, the method comprises: translating coordinates of the at least one virtual box to shifts of the XYZ stage platform that place a center of the sample area in line with an x-ray axis; using a size of the at least one virtual box to define a collimator jaw width in X and Y directions based on a source-to-skin distance specified in the irradiation treatment plan; obtaining sensor data for real-time physiological and/or ambient environment telemetry for an irradiation chamber and/or the sample holder; and/or varying an irradiation regiment during beam delivery based on the sensor data.

In those or other scenarios, the method comprises: establishing, by the processor, a communication link with the sample holder and a processor of the cabinet irradiator via the XYZ stage platform; obtaining, by the processor, a unique identifier from a circuit of the sample holder; using, by the processor, the unique identifier to obtain geometric dimensions and characteristics of the sample holder from a datastore; and/or using artificial intelligence to (i) determine characteristics of the at least one sample based on images captured by the camera or another imaging modality, and (ii) select an irradiation field of a plurality of irradiation fields based on the determined characteristics of the at least one sample.

The present solution also concerns a system comprising a processor and a non-transitory computer readable medium. The computer readable medium comprises one or more programming instructions that when executed by the processor, cause the processor to: detect when a sample holder has been received in a receptacle of an XYZ stage platform of a cabinet irradiator, wherein at least one sample is disposed on the sample holder; control the XYZ stage platform to move the sample holder under a camera such that an optical axis is centered over a sample area of the sample holder; display a top-down image of the at least one sample in a first graphical user interface along with at least one virtual box having an adjustable size and being movable relative to the top-down image; receive a user input specifying at least one area of the at least one sample that is to be irradiated via movement and or resizing of the at least one virtual box; receive a user input selecting or defining an irradiation treatment plan; and cause the cabinet irradiator to irradiate the at least one sample in accordance with the irradiation treatment plan.

In some scenarios, the sample(s) is(are) living. The method also comprises supplying gas from the cabinet irradiator to the sample holder for distribution to the at least one sample. The gas can include, but is not limited to, anesthesia or oxygen. The sample holder may comprise a gas distribution tube connected to a gas distribution tube of the cabinet irradiator via a gas port of the XYZ stage platform. The irradiation treatment plan may be selected from a plurality of pre-defined irradiation treatment plans with different values for at least one of a beam definition, a dose, and a source-to-skin distance. The irradiation treatment plan may comprise a custom or customized irradiation treatment plan.

In those or other scenarios, the processor is further caused to: provide a widget on the first graphical user interface that is configured to allow adjustment of one or more treatment plan parameters; and/or receive another user input adjusting the one or more treatment plan parameters. The treatment plan parameters can include, but are not limited to, a beam definition, a dose, and/or a source-to-skin distance.

In those or other scenarios, the processor is further caused to: display a second graphical user interface configured to assist a user with identification of desired parameters for the custom irradiation treatment plan; receive another user input adjusting the treatment plan parameter(s) to define a first irradiation treatment plan scenario; and present an illustration in the second graphical user interface that shows a visual representation of how far radiation is expected to travel inside the at least one sample in the first irradiation treatment plan scenario. The second graphical user interface can include, but is not limited to, widget(s) to facilitate adjustment of at least one treatment plan parameter to selectively define different irradiation treatment plan scenarios. The treatment plan parameter(s) can include, but is(are) not limited to, a source-to-skin distance.

In those or other scenarios, the processor is further caused to: receive another user input adjusting the at least one treatment plan parameter to define a different second irradiation treatment plan scenario; dynamically modify or replace the illustration in the second graphical user interface to show a visual representation of how far radiation is expected to travel inside the at least one sample in the different second irradiation treatment plan scenario; receive another user input specifying the desired parameters for the custom irradiation treatment plan; automatically determine a source-to-skin distance for the irradiation treatment plan based on a desired field size, a minimum surface-to-skin distance limited by a filter wheel and a collimator of the cabinet irradiator, and a maximum allowed surface-to-skin distance limited by dimensions of an irradiation chamber and XYZ stage travel; set the source-to-skin distance to the minimum surface-to-skin distance when the desired field width is smaller than a maximum field width; set the source-to-skin distance to (SSDₘᵢₙ*tan(θ_{Collimator}), when the desired field width is larger than a maximum field width; translate coordinates of the at least one virtual box to shifts of the XYZ stage platform that place a center of the sample area in line with an x-ray axis; use a size of the at least one virtual box to define a collimator jaw width in X and Y directions based on a source-to-skin distance specified in the irradiation treatment plan; obtain sensor data for real-time physiological and/or ambient environment telemetry for an irradiation chamber and or the sample holder; and/or vary an irradiation regiment during beam delivery based on the sensor data.

In those or other scenarios, the processor is further caused to: establish a communication link with the sample holder and a processor of the cabinet irradiator via the XYZ stage platform; obtain a unique identifier from a circuit of the sample holder; use the unique identifier to obtain geometric dimensions and characteristics of the sample holder from a datastore; and/or use artificial intelligence to (i) determine characteristics of the at least one sample based on images captured by the camera or another imaging modality, and (ii) select an irradiation field of a plurality of irradiation fields based on the determined characteristics of the at least one sample.

The present solution further concerns a sample holder (e.g., sample holder 202 of FIGS. 2 and/or 1414 of FIG. 14). The sample holder may be used with an irradiation cabinet (e.g., irradiation cabinet 100 of FIG. 1). The sample holder may be sized and shaped to be received in an irradiation chamber of the irradiation cabinet. Sample(s) disposed on the sample holder may be exposed to radiation when disposed inside the irradiation cabinet.

The sample holder comprises: a planar irradiation bed (e.g., irradiation bed 1104 of FIG. 11A) comprising a plurality of apertures (e.g., apertures 1108 of FIG. 11A) formed in a grid pattern; at least one accessory (e.g., accessory 1110, 1112 and/or 1114 of FIG. 11A) comprising at least one end configured to be inserted into and removed from each of the plurality of apertures; a first connector (e.g., connector 1124 of FIG. 11A) coupled to the planar irradiation bed and configured to mate with a second connector (e.g., connector 1010 of FIG. 10) of a receptacle of an irradiation cabinet (e.g., irradiation cabinet 100 of FIG. 1) to establish an electrical connection between a circuit (e.g., circuit 1130 of FIG. 11A) of the sample holder and a circuit (e.g., computing device 300 of FIG. 3) of the irradiation cabinet; and a gas delivery tube configured to receive a gas from the irradiation cabinet and deliver the gas to a sample area of the planar irradiation bed.

The accessory can include, but is not limited to, a gas mask (e.g. gas mask 1112 of FIG. 11A), a shielding plate (e.g., a shielding plate 1110 of FIG. 11A), a restraining member (e.g., a restraining member 1114 of FIG. 11A), and/or an environmental control component (e.g., a sensor of circuit 1130 of FIG. 11A). The gas mask may be configured to direct and retain at least some of the gas within the sample area of the irradiation bed. The shielding plate may be configured to reduce or shield secondary scatter radiation from traveling from a first portion of the sample area to an adjacent second portion of the sample area of the irradiation bed. The restraining member may be configured to restrain at least one sample in the sample area of the irradiation bed.

In those or other scenarios, the sample holder may comprise a sidewall protruding from the planar irradiation bed. The sidewall may comprise tube connection port(s) (e.g., tube connector port(s) 1134 of FIG. 11A) to connect a gas delivery tube of the irradiation cabinet to the gas mask coupled to the irradiation bed. The circuit of the sample holder can include, but is not limited to, a datastore storing a unique identifier for the sample holder, a temperature sensor, a humidity sensor, an oxygen sensor, and/or a valve for controlling a flow of the gas to the sample area. The unique identifier may comprise an index to retrieve information from a remote datastore specifying geometric dimensions of the sample holder, a material of the sample holder, and radiation characteristics of the sample holder. The grid pattern of the apertures may facilitate a translation of virtual box coordinates in a graphical user interface to shifts of an XYZ stage platform of the irradiation cabinet to place the sample area in line with an x-ray axis.

In those or other scenarios, the irradiation bed may be configured to compensate for x-ray beam non-uniformity without reduction of a dose rate arising from flattening filters of the irradiation cabinet. The irradiation bed comprises a variable height thereacross. Additionally or alternatively, a height of a portion of the sample area is adjustable to facilitate different surface-to-skin distances between an x-ray tube of the irradiation cabinet and surfaces of samples where an x-ray beam is to enter.

The present solution also concerns an irradiation system, comprising: a vertical wheel (e.g., wheel 1402 of FIG. 14) structurally supported by a stand (e.g., stand 1404 of FIG. 14) such that the wheel can rotate; and a plurality of sample holders (e.g., sample holders 1414 of FIG. 4) suspended on an outer edge of the vertical wheel in a manner that maintains a horizontal alignment of each of the sample holders with a surface on which the stand sits. A surface-to-skin distance parameter for an irradiation treatment plan is defined by a distance between an x-ray tube and a sample disposed in a top most one of the sample holders. The sample holders may be configured to be selectively moved in and out of alignment with an x-ray axis via rotation of the vertical wheel.

The vertical wheel comprises a first circular rim (e.g., rim 1410 of FIG. 14) and a second circular rim (e.g., rim 1412 of FIG. 14) parallel to the first circular rim. An axis of a cylinder defined by the first and second rims may extend parallel to or perpendicular to an irradiation chamber floor when at least one of the plurality of sample holders is being used to facilitate irradiation of a sample.

The irradiation system may also comprise at least one shielding plate (e.g., shielding plate 1110 of FIG. 11A) configured to be coupled to a sample holder (e.g., sample holder 202 of FIG. 11A) of the plurality of sample holders and configured to reduce or shield secondary scatter radiation from traveling from a first portion of the sample area of the sample holder to an adjacent second portion of the sample area of the sample holder.

Each sample holder may comprise: a planar irradiation bed (e.g., irradiation bed 1104 of FIG. 11A) comprising a plurality of apertures (e.g., apertures 1108 of FIG. 11A) formed in a grid pattern; at least one accessory (e.g., accessory 1110, 1112 and/or 1114 of FIG. 11A) comprising at least one end configured to be inserted into and removed from each of the plurality of apertures; a first connector (e.g., connector 1124 of FIG. 11A) coupled to the planar irradiation bed and configured to mate with a second connector (e.g., connector 1010 of FIG. 10) of a receptacle of an irradiation cabinet (e.g., irradiation cabinet 100 of FIG. 1) to establish an electrical connection between a circuit (e.g., circuit 1130 of FIG. 11A) of the sample holder and a circuit (e.g., computing device 300 of FIG. 3) of the irradiation cabinet; and a gas delivery tube configured to receive a gas from the irradiation cabinet and deliver the gas to a sample area of the planar irradiation bed.

The accessory(ies) can include, but is(are) not limited to, a gas mask (e.g., gas mask 1112 of FIG. 11A), a shielding plate (e.g., shielding plate 1110 of FIG. 11A), a restraining member (e.g., restraining member 1114 of FIG. 11A), and/or an environmental control component (e.g., circuit 1130 or component thereof of FIG. 11A). The gas mask may be configured to direct and retain at least some of the gas within the sample area of the irradiation bed. The shielding plate may be configured to reduce or shield secondary scatter radiation from traveling from a first portion of the sample area to an adjacent second portion of the sample area of the irradiation bed. The restraining member may be configured to restrain at least one sample in the sample area of the irradiation bed. A sidewall may protrude from the planar irradiation bed. The sidewall may comprise tube connection port(s) (e.g., tube connection port(s) 1134 of FIG. 11A) to connect a gas delivery tube of the irradiation cabinet to the gas mask coupled to the irradiation bed.

The circuit of the sample holder can include, but is not limited to, a datastore storing a unique identifier for the sample holder, a temperature sensor, a humidity sensor, an oxygen sensor, and/or a valve for controlling a flow of the gas to the sample area. The unique identifier may include, but is not limited to, an index to retrieve information from a remote datastore specifying geometric dimensions of the sample holder, a material of the sample holder, and radiation characteristics of the sample holder.

The irradiation bed may be configured to compensate for x-ray beam non-uniformity without reduction of a dose rate arising from flattening filters of the irradiation cabinet. The irradiation bed may have a variable height thereacross. A height of a portion of the sample area may be adjustable to facilitate different surface-to-skin distances between an x-ray tube of the irradiation cabinet and surfaces of samples where an x-ray beam is to enter. The grid pattern of the apertures may facilitate a translation of virtual box coordinates in a graphical user interface to shifts of an XYZ stage platform of the irradiation cabinet to place the sample area in line with an x-ray axis.

The present solution also concerns implementing systems and methods for operating an irradiation system. The methods comprise: receiving, by in an irradiation chamber of an irradiator, a wheel structurally supported by a stand; suspending a plurality of sample holders on an outer edge of the wheel in a manner that maintains a horizontal alignment of each of the sample holders with a surface of the irradiation chamber on which the stand sits; exposing a first sample disposed on a first sample holder of the plurality of sample holders to radiation with a uniform distribution and at a specific dose rate, the first sample holder being closest to an x-ray tube of the irradiator than other ones of the plurality of sample holders; discontinuing radiation exposure to the first sample by rotating the wheel within the irradiation chamber so as to move the first sample holder away from the x-ray tube and out of alignment with an x-ray axis; continue rotating the wheel within the irradiation chamber so as to move another second sample holder closer to the x-ray tube and into alignment with the x-ray axis; and exposing a second sample disposed on the second sample holder of the plurality of sample holders to the radiation with the uniform distribution and at the specific dose rate.

A surface-to-skin distance parameter for an irradiation treatment plan is defined by a distance between the x-ray tube of the irradiator and a sample disposed in a top most one of the plurality of sample holders. The methods may also comprise modifying a value of the surface-to-skin distance parameter by adjusting a height of a portion of a sample area of the first sample holder.

Additionally or alternatively, the methods may also comprise: using at least one shielding plate coupled to the first sample holder to reduce or shield secondary scatter radiation from traveling from a first portion of a sample area of the first sample holder to an adjacent second portion of the sample area of the first sample holder; connecting a gas delivery tube of the irradiator to a gas delivery tube of the first sample holder via the wheel; receiving at the first sample holder gas from the irradiator; using a valve of the first sample holder to control a flow of gas to a sample area of the first sample holder; using a gas mask coupled to the first sample holder to direct and retain at least some of the gas within the sample area of the first sample holder; using a restraining member coupled to the first sample holder to restrain the first sample in a sample area of the first sample holder; and/or performing operations by a circuit of the first sample holder to detect characteristics of an internal environment of the irradiator.

Additionally or alternatively, the methods may also comprise: supplying power to the circuit of the first sample holder via an electrical connection between the first sample holder and the wheel and via an electrical connection between the wheel and a circuit of the irradiator; using a grid pattern of a plurality of apertures of the first sample holder to facilitate a translation of virtual box coordinates in a graphical user interface of the irradiator to shifts of an XYZ stage platform of the irradiator; and/or using the shifts of an XYZ stage platform to align the first sample with the x-ray axis.

The x-ray tube 204 of the present solution may comprise an x-ray source described in U.S. Provisional Application Ser. No. 63/753,109 which was filed on Feb. 3, 2025. The content of this U.S. Provisional Application is incorporated herein by reference in its entirety. The x-ray source of the cabinet irradiator 100 may comprise an elongated x-ray tube 204 (having an outer surface, an open interior, and first and second ends), a cathode, and an anode. The cathode and anode may be in the open interior. The cathode may be nearest the first end. The anode may be nearest the second end. The cathode and anode may be separated by a length L. A target in the open interior is nearer the second end than the first end. The target comprises a material that when impacted by the electron beam generates Bremsstrahlung x-ray photons. The cathode and the target define an electron beam path for an electron beam. The x-ray source also comprises a voltage multiplier circuit and a field balancing circuit. Capacitors and diodes of the voltage multiplier circuit and the field balancing circuit are positioned radially outward from the—outer surface of the elongated x-ray tube to a distance that is less than or equal to the distance L, and are axially distributed between the cathode and the anode. During operation of the x-ray source the voltage multiplier circuit produces a voltage multiplier electromagnetic field. The field balancing circuit produces a field balancing electromagnetic field that counteracts the voltage multiplier electromagnetic field such that the electron beam path is unaffected by the voltage multiplier electromagnetic field.

The voltage multiplier circuit and the field balancing circuit may be positioned radially outward from the electron beam path from the outer surface of the elongated x-ray tube to a distance that is less than the distance 0.500 L or a distance that is less than the distance 0.250 L. The x-ray the voltage multiplier circuit and the field balancing circuit may be attached to the outer surface of the elongated x-ray tube. The field balancing circuit may comprise a voltage meter circuit comprising a series of resistors electrically connected between the anode and the cathode for measuring the voltage between the anode and the cathode. A portion of the voltage multiplier circuit may comprise the capacitors and diodes of the voltage multiplier circuit is affixed to the outer surface of the elongated x-ray tube. The field balancing circuit may be affixed to the outer surface of the elongated x-ray tube. The x-ray tube may have longitudinal half sections, and the voltage multiplier circuit and field balancing circuit may be positioned radially outward from different ones of the longitudinal half sections. The target may include the anode. The target may be electrically separated from the anode. There may be no EMI shield between the voltage multiplier circuit and the field balancing circuit and the x-ray tube.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices. A computer program product is a memory device with programming instructions stored on it.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

The described features, advantages and characteristics disclosed herein may be combined in any suitable manner. One skilled in the relevant art will recognize, in light of the description herein, that the disclosed systems and/or methods can be practiced without one or more of the specific features. In other instances, additional features and advantages may be recognized in certain scenarios that may not be present in all instances.

Although the systems and methods have been illustrated and described with respect to one or more implementations, equivalent alterations and modifications will occur to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application. Thus, the breadth and scope of the disclosure herein should not be limited by any of the above descriptions. Rather, the scope of the invention should be defined in accordance with the following claims and their equivalents.

1. A method for operating a cabinet irradiator, comprising:
receiving a sample holder in a receptacle of an XYZ stage platform of the cabinet irradiator, wherein at least one sample is disposed on the sample holder;
translating coordinates of the at least one virtual box to shifts of the XYZ stage platform that place a center of the sample area in line with an x-ray axis;
controlling, by a processor, the XYZ stage platform to move the sample holder under a camera such that an optical axis is centered over a sample area of the sample holder;
displaying, by the cabinet irradiator, a top-down image of the at least one sample in a first graphical user interface along with at least one virtual box having an adjustable size and being movable relative to the top-down image;
receiving, by the processor, a user input specifying at least one area of the at least one sample that is to be irradiated via movement and or resizing of the at least one virtual box;
receiving, by the processor, a user input selecting or defining an irradiation treatment plan; and
irradiating, by the cabinet irradiator, the at least one sample in accordance with the irradiation treatment plan.

2. The method according to claim 1, wherein the at least one sample is living and the method further comprises supplying gas from the cabinet irradiator to the sample holder for distribution to the at least one sample.

3. The method according to claim 2, wherein the gas comprises anesthesia or oxygen.

4. The method according to claim 2, wherein the sample holder comprises a gas distribution tube connected to a gas distribution tube of the cabinet irradiator via a gas port of the XYZ stage platform.

5. The method according to claim 1, wherein the irradiation treatment plan is selected from a plurality of pre-defined irradiation treatment plans with different values for at least one of a beam definition, a dose, and a source-to-skin distance.

6. The method according to claim 1, wherein the irradiation treatment plan comprises a custom irradiation treatment plan.

7. The method according to claim 6, further comprising:
providing a widget on the first graphical user interface that is configured to allow adjustment of one or more treatment plan parameters, wherein the treatment plan parameters comprise at least a beam definition, a dose, and a source-to-skin distance; and
receiving another user input adjusting the one or more treatment plan parameters.

8. A method for operating a cabinet irradiator, comprising:
receiving a sample holder in a receptacle of an XYZ stage platform of the cabinet irradiator, wherein at least one sample is disposed on the sample holder;
controlling, by a processor, the XYZ stage platform to move the sample holder under a camera such that an optical axis is centered over a sample area of the sample holder;
displaying, by the cabinet irradiator, a top-down image of the at least one sample in a first graphical user interface along with at least one virtual box having an adjustable size and being movable relative to the top-down image;
receiving, by the processor, a user input specifying at least one area of the at least one sample that is to be irradiated via movement and or resizing of the at least one virtual box;
receiving, by the processor, a user input selecting or defining an irradiation treatment plan; and
irradiating, by the cabinet irradiator, the at least one sample in accordance with the irradiation treatment plan;
wherein the irradiation treatment plan comprises a custom irradiation treatment plan; and
wherein the method further comprises:
displaying a second graphical user interface configured to assist a user with identification of desired parameters for the custom irradiation treatment plan, the second graphical user interface comprising at least one widget to facilitate adjustment of at least one treatment plan parameter to selectively define different irradiation treatment plan scenarios;
receiving another user input adjusting the at least one treatment plan parameter to define a first irradiation treatment plan scenario; and
presenting an illustration in the second graphical user interface that shows a visual representation of how far radiation is expected to travel inside the at least one sample in the first irradiation treatment plan scenario.

9. The method according to claim 8, wherein the at least one treatment plan parameter comprises a source-to-skin distance.

10. The method according to claim 8, further comprising:
receiving another user input adjusting the at least one treatment plan parameter to define a different second irradiation treatment plan scenario; and
dynamically modifying or replacing the illustration in the second graphical user interface to show a visual representation of how far radiation is expected to travel inside the at least one sample in the different second irradiation treatment plan scenario.

11. The method according to claim 10, further comprising receiving another user input specifying the desired parameters for the custom irradiation treatment plan.

12. A method for operating a cabinet irradiator, comprising:
receiving a sample holder in a receptacle of an XYZ stage platform of the cabinet irradiator, wherein at least one sample is disposed on the sample holder;
controlling, by a processor, the XYZ stage platform to move the sample holder under a camera such that an optical axis is centered over a sample area of the sample holder;
displaying, by the cabinet irradiator, a top-down image of the at least one sample in a first graphical user interface along with at least one virtual box having an adjustable size and being movable relative to the top-down image;
receiving, by the processor, a user input specifying at least one area of the at least one sample that is to be irradiated via movement and or resizing of the at least one virtual box;
receiving, by the processor, a user input selecting or defining an irradiation treatment plan;
irradiating, by the cabinet irradiator, the at least one sample in accordance with the irradiation treatment plan; and
automatically determining, by the processor, a source-to-skin distance for the irradiation treatment plan based on a desired field size, a minimum surface-to-skin distance limited by a filter wheel and a collimator of the cabinet irradiator, and a maximum allowed surface-to-skin distance limited by dimensions of an irradiation chamber and XYZ stage travel.

13. The method according to claim 12, further comprising setting the source-to-skin distance to the minimum surface-to-skin distance when the desired field width is smaller than a maximum field width.

14. The method according to claim 12, further comprising setting the source-to-skin distance to (SSDmin*tan(8collimator), when the desired field width is larger than a maximum field width.

15. (canceled)

16. A method for operating a cabinet irradiator, comprising:
receiving a sample holder in a receptacle of an XYZ stage platform of the cabinet irradiator, wherein at least one sample is disposed on the sample holder;
controlling, by a processor, the XYZ stage platform to move the sample holder under a camera such that an optical axis is centered over a sample area of the sample holder;
displaying, by the cabinet irradiator, a top-down image of the at least one sample in a first graphical user interface along with at least one virtual box having an adjustable size and being movable relative to the top-down image;
receiving, by the processor, a user input specifying at least one area of the at least one sample that is to be irradiated via movement and or resizing of the at least one virtual box;
receiving, by the processor, a user input selecting or defining an irradiation treatment plan;
irradiating, by the cabinet irradiator, the at least one sample in accordance with the irradiation treatment plan; and
using a size of the at least one virtual box to define a collimator jaw width in X and Y directions based on a source-to-skin distance specified in the irradiation treatment plan.

17. The method according to claim 1, further comprising obtaining sensor data for real-time physiological and/or ambient environment telemetry for an irradiation chamber and or the sample holder.

18. The method according to claim 17, further comprising vary an irradiation regiment during beam delivery based on the sensor data.

19. A method for operating a cabinet irradiator, comprising:
receiving a sample holder in a receptacle of an XYZ stage platform of the cabinet irradiator, wherein at least one sample is disposed on the sample holder;
controlling, by a processor, the XYZ stage platform to move the sample holder under a camera such that an optical axis is centered over a sample area of the sample holder;
displaying, by the cabinet irradiator, a top-down image of the at least one sample in a first graphical user interface along with at least one virtual box having an adjustable size and being movable relative to the top-down image;
receiving, by the processor, a user input specifying at least one area of the at least one sample that is to be irradiated via movement and or resizing of the at least one virtual box;
receiving, by the processor, a user input selecting or defining an irradiation treatment plan;
irradiating, by the cabinet irradiator, the at least one sample in accordance with the irradiation treatment plan;
establishing, by the processor, a communication link with the sample holder and a processor of the cabinet irradiator via the XYZ stage platform; and
obtaining, by the processor, a unique identifier from a circuit of the sample holder; and using, by the processor, the unique identifier to obtain geometric dimensions and characteristics of the sample holder from a datastore.

20. The method according to claim 1, further comprising using artificial intelligence to (i) determine characteristics of the at least one sample based on images captured by the camera or another imaging modality, and (ii) select an irradiation field of a plurality of irradiation fields based on the determined characteristics of the at least one sample.

21. A system, comprising:
a processor; and
a non-transitory computer readable medium comprising one or more programming instructions that when executed by the processor, cause the processor to:
detect when a sample holder has been received in a receptacle of an XYZ stage platform of a cabinet irradiator, wherein at least one sample is disposed on the sample holder;
translate coordinates of the at least one virtual box to shifts of the XYZ stage platform that place a center of the sample area in line with an x-ray axis;
control the XYZ stage platform to move the sample holder under a camera such that an optical axis is centered over a sample area of the sample holder;
display a top-down image of the at least one sample in a first graphical user interface along with at least one virtual box having an adjustable size and being movable relative to the top-down image;
receive a user input specifying at least one area of the at least one sample that is to be irradiated via movement and or resizing of the at least one virtual box;
receive a user input selecting or defining an irradiation treatment plan; and
cause the cabinet irradiator to irradiate the at least one sample in accordance with the irradiation treatment plan.

22. The system according to claim 21, wherein the at least one sample is living and the method further comprises supplying gas from the cabinet irradiator to the sample holder for distribution to the at least one sample.

23. The system according to claim 22, wherein the gas comprises anesthesia or oxygen.

24. The system according to claim 22, wherein the sample holder comprises a gas distribution tube connected to a gas distribution tube of the cabinet irradiator via a gas port of the XYZ stage platform.

25. The system according to claim 21, wherein the irradiation treatment plan is selected from a plurality of pre-defined irradiation treatment plans with different values for at least one of a beam definition, a dose, and a source-to-skin distance.

26. The system according to claim 21, wherein the irradiation treatment plan comprises a custom irradiation treatment plan.

27. The system according to claim 26, wherein the processor is further caused to:
provide a widget on the first graphical user interface that is configured to allow adjustment of one or more treatment plan parameters, wherein the treatment plan parameters comprise at least a beam definition, a dose, and a source-to-skin distance; and
receive another user input adjusting the one or more treatment plan parameters.

28. A system, comprising:
a processor; and
a non-transitory computer readable medium comprising one or more programming instructions that when executed by the processor, cause the processor to:
detect when a sample holder has been received in a receptacle of an XYZ stage platform of a cabinet irradiator, wherein at least one sample is disposed on the sample holder;
control the XYZ stage platform to move the sample holder under a camera such that an optical axis is centered over a sample area of the sample holder;
display a top-down image of the at least one sample in a first graphical user interface along with at least one virtual box having an adjustable size and being movable relative to the top-down image;
receive a user input specifying at least one area of the at least one sample that is to be irradiated via movement and or resizing of the at least one virtual box;
receive a user input selecting or defining an irradiation treatment plan; and
cause the cabinet irradiator to irradiate the at least one sample in accordance with the irradiation treatment plan;
wherein the irradiation treatment plan comprises a custom irradiation treatment plan;
wherein the processor is further caused to:
display a second graphical user interface configured to assist a user with identification of desired parameters for the custom irradiation treatment plan, the second graphical user interface comprising at least one widget to facilitate adjustment of at least one treatment plan parameter to selectively define different irradiation treatment plan scenarios;
receive another user input adjusting the at least one treatment plan parameter to define a first irradiation treatment plan scenario; and
present an illustration in the second graphical user interface that shows a visual representation of how far radiation is expected to travel inside the at least one sample in the first irradiation treatment plan scenario.

29. The system according to claim 28, wherein the at least one treatment plan parameter comprises a source-to-skin distance.

30. The system according to claim 28, wherein the processor is further caused to:
receive another user input adjusting the at least one treatment plan parameter to define a different second irradiation treatment plan scenario; and
dynamically modify or replace the illustration in the second graphical user interface to show a visual representation of how far radiation is expected to travel inside the at least one sample in the different second irradiation treatment plan scenario.

31. The system according to claim 30, wherein the processor is further caused to receive another user input specifying the desired parameters for the custom irradiation treatment plan.

32. A system, comprising:
a processor; and
a non-transitory computer readable medium comprising one or more programming instructions that when executed by the processor, cause the processor to:
detect when a sample holder has been received in a receptacle of an XYZ stage platform of a cabinet irradiator, wherein at least one sample is disposed on the sample holder;
control the XYZ stage platform to move the sample holder under a camera such that an optical axis is centered over a sample area of the sample holder;
display a top-down image of the at least one sample in a first graphical user interface along with at least one virtual box having an adjustable size and being movable relative to the top-down image;
receive a user input specifying at least one area of the at least one sample that is to be irradiated via movement and or resizing of the at least one virtual box;
receive a user input selecting or defining an irradiation treatment plan; and
cause the cabinet irradiator to irradiate the at least one sample in accordance with the irradiation treatment plan
wherein the processor is further caused to automatically determine a source-to-skin distance for the irradiation treatment plan based on a desired field size, a minimum surface-to-skin distance limited by a filter wheel and a collimator of the cabinet irradiator, and a maximum allowed surface-to-skin distance limited by dimensions of an irradiation chamber and XYZ stage travel.

33. The system according to claim 32, wherein the processor is further caused to set the source-to-skin distance to the minimum surface-to-skin distance when the desired field width is smaller than a maximum field width.

34. The system according to claim 32, wherein the processor is further caused to set the source-to-skin distance to (SSDmin*tan(8collimator), when the desired field width is larger than a maximum field width.

35. (canceled)

36. A system, comprising:
a processor; and
a non-transitory computer readable medium comprising one or more programming instructions that when executed by the processor, cause the processor to:
detect when a sample holder has been received in a receptacle of an XYZ stage platform of a cabinet irradiator, wherein at least one sample is disposed on the sample holder;
control the XYZ stage platform to move the sample holder under a camera such that an optical axis is centered over a sample area of the sample holder;
display a top-down image of the at least one sample in a first graphical user interface along with at least one virtual box having an adjustable size and being movable relative to the top-down image;
receive a user input specifying at least one area of the at least one sample that is to be irradiated via movement and or resizing of the at least one virtual box;
receive a user input selecting or defining an irradiation treatment plan; and
cause the cabinet irradiator to irradiate the at least one sample in accordance with the irradiation treatment plan
wherein the processor is further caused to use a size of the at least one virtual box to define a collimator jaw width in X and Y directions based on a source-to-skin distance specified in the irradiation treatment plan.

37. The system according to claim 21, wherein the processor is further caused to obtain sensor data for real-time physiological and/or ambient environment telemetry for an irradiation chamber and or the sample holder.

38. The system according to claim 37, wherein the processor is further caused to vary an irradiation regiment during beam delivery based on the sensor data.

39. A system, comprising:
a processor; and
a non-transitory computer readable medium comprising one or more programming instructions that when executed by the processor, cause the processor to:
detect when a sample holder has been received in a receptacle of an XYZ stage platform of a cabinet irradiator, wherein at least one sample is disposed on the sample holder;
control the XYZ stage platform to move the sample holder under a camera such that an optical axis is centered over a sample area of the sample holder;
display a top-down image of the at least one sample in a first graphical user interface along with at least one virtual box having an adjustable size and being movable relative to the top-down image;
receive a user input specifying at least one area of the at least one sample that is to be irradiated via movement and or resizing of the at least one virtual box;
receive a user input selecting or defining an irradiation treatment plan; and
cause the cabinet irradiator to irradiate the at least one sample in accordance with the irradiation treatment plan;
wherein the processor is further caused to:
establish a communication link with the sample holder and a processor of the cabinet irradiator via the XYZ stage platform;
obtain a unique identifier from a circuit of the sample holder; and
use the unique identifier to obtain geometric dimensions and characteristics of the sample holder from a datastore.

40. The system according to claim 21, wherein the processor is further caused to use artificial intelligence to (i) determine characteristics of the at least one sample based on images captured by the camera or another imaging modality, and (ii) select an irradiation field of a plurality of irradiation fields based on the determined characteristics of the at least one sample.
